(19) United States
(12) Patent Application Publication
(10) Pub. No.: US 2018/0333426 A1
(43) Pub. Date: Nov. 22, 2018

(54) NUTRITIONAL COMPOSITION WITH HUMAN MILK OLIGOSACCHARIDES AND USES THEREOF

(71) Applicant: Mead Johnson Nutrition Company, Chicago, IL (US)

(72) Inventors: Maciej Chichlowski, Fair Oaks, CA (US); Brian Berg, Evansville, IN (US); Zihua Ao, Newburgh, IN (US); Rosaline Waworuntu, Evansville, IN (US)

(21) Appl. No.: 15/597,575

(22) Filed: May 17, 2017

(51) Int. Cl. A61K 31/702; A23L 33/00; A23L 33/135; A23L 33/12; A23L 33/17; A23L 33/125; A23L 33/10; A61K 35/20; A61K 31/70; A61K 38/00; A61K 31/716; A61K 35/747; A61K 31/202; A61K 38/40

(52) U.S. Cl. CPC A61K 31/702; A61K 2035/11; A23L 33/30; A23L 33/135; A23L 33/12; A23L 33/17; A23L 33/125; A23L 33/10; A61K 35/20; A61K 31/70; A61K 38/00; A61K 31/716; A61K 35/747; A61K 31/202; A61K 38/40; A23V 2002/00; A23Y 2220/73; A23L 33/40

(57) ABSTRACT

The present disclosure generally relates to pediatric nutritional compositions including galacto-oligosaccharide and human milk oligosaccharide or a precursor thereof. Further, the nutritional compositions may include GOS in combination with a first human milk oligosaccharide or precursor thereof and a second human milk oligosaccharide or precursor thereof. The disclosed nutritional compositions advantageously modify gut microbiome and improve select markers of immunity, brain structure, and gut function.

### TECHNICAL FIELD

The present disclosure generally provides nutritional compositions containing human milk oligosaccharides (“HMO”) that are useful for improving health via beneficial action in the developing gut. In some embodiments, the human milk oligosaccharides include N-acetylglucosamine and L-fucose and the nutritional composition may further comprise additional prebiotics, such as galacto-oligosaccharides. Further disclosed are methods for improving and/or creating a beneficial gut microbiome profile, and also for promoting the growth of beneficial microbiota in the gastrointestinal tract of pediatric subjects comprising administering to a subject the disclosed nutritional composition. Further, provided are methods for increasing sialic acid in neurological tissues, including brain tissues in target subjects. In some embodiments, the target subject may be pediatric subjects including infants and preterm infants.

### BACKGROUND

Human infant gut microbiota is rapidly established in the first few weeks following birth. Gut microbiota development in infants is understood to be initiated by exposure to maternal and environmental bacteria during birth. Further development of gut microbiota is affected by a newborn infant's diet. Whether the infant is breast fed or formula fed has a strong influence on the intestinal bacterial population and composition. Human milk contains numerous macro and micronutrient components, the identity and function of which are still being discovered and studied. Among these components, human milk oligosaccharides are believed to play an important role in the growth of beneficial bacteria in infants. In the breast fed infant, for example, Bifidobacterium species dominate among intestinal bacteria, while Streptococcus species and Lactobacillus species are less common. In contrast, the microbiota of formula fed infants is more diverse. The species of Bifidobacterium in the stools of breast fed and formula fed infants vary as well. Bifidobacterium species are generally considered beneficial bacteria and are known to protect against colonization by pathogenic bacteria.

Gut microbiota is also important for healthy brain function, and it is believed that gut microbiota communicate with the brain via the gut-brain axis, and thus have an impact on brain development and function. More specifically, gut microbiota interact with enteric and central nervous systems via neural, hormonal, and immune links. Brain development and growth exceeds that of any other organ or body tissue, reaching its peak at 26 weeks of gestation and continuing at a rapid rate throughout the first three years of life. Sub-optimal nutrition during this phase may have irreversible consequences for cognitive function.

Accordingly, there is a need to provide nutritional compositions, such as infant formulas, that promote the growth of healthy gut microbiota, and promote a healthy gut-brain axis. Further, there is a need for nutritional compositions that address the problem of leaky gut by directly targeting tight junction expression and cytokine production in addition to beneficially modulating the microbiome composition. Such compositions may provide improved cognitive development in infants and children, and thus provide lifelong brain benefits. The present disclosure addresses this need by providing nutritional compositions comprising HMO and, in some embodiments, a prebiotic.

### BRIEF SUMMARY

The present disclosure is directed, in some embodiments, to a nutritional composition comprising HMO or one or more precursors thereof, and, in some embodiments, GOS and/or PDX. In certain embodiments, the HMO comprise N-acetylglucosamine and/or L-fucose. While not being bound by any particular theory, it is believed that HMO in combination with GOS, may act synergistically when included in nutritional compositions, such as infant formulas, to promote the growth and/or function of beneficial gut microbiota, thereby stimulating the gut-brain axis. Such compositions may therefore promote healthy cognitive development in infants and children. More specifically, the nutritional compositions provided herein comprise in some embodiments: (i) a protein source, (ii) a lipid source, (iii) a carbohydrate source, (iv) HMO or a precursor thereof, and (v) a prebiotic comprising galacto-oligosaccharide.

In some embodiments, the nutritional composition comprises a first oligosaccharide, comprising GOS, in a concentration of from about 1 mg/mL to about 6 mg/mL; a second oligosaccharide comprising an HMO or precursor thereof selected from 3′-sialyllactose, 6′-sialyllactose, lacto-N-biose, lacto-N-neotetraose, lacto-N-tetraose, and combinations thereof, in a concentration of from about 0.5 mg/mL to about 10 mg/mL; and a third HMO or precursor thereof selected from N-acetylglucosamine and/or L-fucose in a concentration of from about 0.1 mg/mL to about 5.0 mg/mL. Indeed, such a synergistic combination would work in a synergistic manner by stimulating the growth and activity of naturally occurring beneficial bacteria, positively affecting gut health, stimulating beneficial bacterial metabolites, supporting epithelial barrier and immune system development and facilitating optimal neuronal development.

Further, administration of the nutritional compositions disclosed herein may improve overall gut function and gut health in target subjects via modulation of gut microbiota. Indeed, administration of the nutritional compositions disclose herein may increase levels of beneficial infant-specific, bacterial strains and lower exposure to antibiotic-resistant microbes. Accordingly, disclosed herein are methods for increasing levels of beneficial bacterial strains in the gut of the target subject. Further, administration of the nutritional compositions disclosed herein may lower microbiome alpha diversity measure (including richness and evenness).

Further disclosed are methods for lowering the exposure to antibiotic-resistant microbes, such as Proteobacteria, in a target subject via administration of the nutritional compositions disclose herein.

In some embodiments, provided are methods for enhancing epithelial barrier function in target subjects via administration of the nutritional compositions disclosed herein.

Also provided herein are methods for beneficially impacting short-chain fatty add concentration in the colon of a target subject via administration of the nutritional compositions disclosed herein.

In some embodiments, provided are methods for reducing the risk of food allergy by promoting the growth of Clostridia (cluster XIVa and IV), via administering the nutritional compositions disclosed herein to the target subject.

The HMO or HMO precursor useful in the present compositions include, but are not limited to, 2′-fucosyllactose, 3′-fucosyllactose, 3′-sialyllactose, 6′-sialyllactose, lacto-N-biose, lacto -N-neotetraose, lacto-N-tetraose, L-fucose, N-acetylglucosamine, or any combination thereof. Precursors of HMO, such as sialic acid, fucose, or a combination thereof, also may be included in the present compositions.

Compositions of the present disclosure may also include, in some embodiments, a source of long chain polyunsaturated fatty acids, such as docosahexaenoic acid (DHA) and/or arachidonic add (ARA), a source of β-glucan, lactoferrin, or any combination thereof.

### BRIEF DESCRIPTION OF THE DRAWINGS

The patent or application file contains at least one drawing executed in color. Copies of this patent or patent application publication with color drawing(s) will be provided by the Office upon request payment of the necessary fee.

FIG. 1 illustrates the metabolic profiles in the feces of C57BL/6 male mice with 3 different blinded diets (A—Control Diet, B—SL+GOS/PDX, C—SL) under stressed (social disruption) or non-stressed conditions. Diet B mice under stress conditions achieved statistical significance (p≤0.05) compared to non-stress mice.

FIG. 2 illustrates the Axial, mean and radial diffusivity; different letters indicate significant differences between groups (P≤0.05) to assess white matter integrity on piglets.

### DETAILED DESCRIPTION

Reference now will be made in detail to the embodiments of the present disclosure, one or more examples of which are set forth herein below. Each example is provided by way of explanation of the nutritional composition of the present disclosure and is not a limitation. In fact, it will be apparent to those skilled in the art that various modifications and variations can be made to the teachings of the present disclosure without departing from the scope or spirit of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only and is not intended as limiting the broader aspects of the present disclosure.

“Nutritional composition” means a substance or formulation that satisfies at least a portion of a subject's nutrient requirements. The terms “nutritional(s),” “nutritional formula(s),” “enteral nutritional(s),” “nutritional composition(s),” and “nutritional supplement(s)” are used interchangeably throughout the present disclosure to refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults, such as women who are lactating or pregnant. In particular embodiments, the nutritional compositions are for pediatric subjects, including infants and children.

The term “enteral” means through or within the gastrointestinal, or digestive, tract. “Enteral administration” includes oral feeding, intragastric feeding, transpyloric administration, or any other administration into the digestive tract.

“Pediatric subject” includes both infants and children, and refers herein to a human that is less than thirteen years of age. In some embodiments, a pediatric subject refers to a human subject that is less than eight years old. In other embodiments, a pediatric subject refers to a human subject between about one and about six years of age or about one and about three years of age. In still further embodiments, a pediatric subject refers to a human subject between about 6 and about 12 years of age.

“Infant” means a subject having an age of not more than about one year and includes infants from about zero to about twelve months. The term infant includes low birth weight infants, very low birth weight infants, and preterm infants. “Preterm” means an infant born before the end of the 37th week of gestation, while “full term” means an infant born after the end of the 37th week of gestation.

“Child” means a subject ranging in age from about twelve months to about thirteen years. In some embodiments, a child is a subject between the ages of one and twelve years old. In other embodiments, the terms “children” or “child” refer to subjects that are between about one and about six years old, between about one and about three years old, or between about seven and about twelve years old. In other embodiments, the terms “children” or “child” refer to any range of ages between about 12 months and about 13 years.

“Children's nutritional product” refers to a composition that satisfies at least a portion of the nutrient requirements of a child. A growing-up milk is an example of a children's nutritional product.

“Infant formula” means a composition that satisfies at least a portion of the nutrient requirements of an infant. In the United States, the content of an infant formula is dictated by the federal regulations set forth at 21 C.F.R. Sections 100, 106, and 107. These regulations define macronutrient, vitamin, mineral, and other ingredient levels in an effort to simulate the nutritional and other properties of human breast milk.

The term “growing-up milk” refers to a broad category of nutritional compositions intended to be used as a part of a diverse diet in order to support the normal growth and development of a child between the ages of about 1 and about 6 years of age.

“Milk-based” means comprising at least one component that has been drawn or extracted from the mammary gland of a mammal. In some embodiments, a milk-based nutritional composition comprises components of milk that are derived from domesticated ungulates, ruminants or other mammals or any combination thereof. Moreover, in some embodiments, milk-based means comprising bovine casein, whey, lactose, or any combination thereof. Further, “milk-based nutritional composition” may refer to any composition comprising any milk-derived or milk-based product known in the art.

“Nutritionally complete” means a composition that may be used as the sole source of nutrition, which would supply essentially all of the required daily amounts of vitamins, minerals, and/or trace elements in combination with proteins, carbohydrates, and lipids.

Indeed, “nutritionally complete” describes a nutritional composition that provides adequate amounts of carbohydrates, lipids, essential fatty acids, proteins, essential amino acids, conditionally essential amino acids, vitamins, minerals and energy required to support normal growth and development of a subject.

Therefore, a nutritional composition that is “nutritionally complete” for a preterm infant will, by definition, provide qualitatively and quantitatively adequate amounts of carbohydrates, lipids, essential fatty acids, proteins, essential amino acids, conditionally essential amino acids, vitamins, minerals, and energy required for growth of the preterm infant.

A nutritional composition that is “nutritionally complete” for a term infant will, by definition, provide qualitatively and quantitatively adequate amounts of all carbohydrates, lipids, essential fatty acids, proteins, essential amino acids, conditionally essential amino acids, vitamins, minerals, and energy required for growth of the term infant.

A nutritional composition that is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

As applied to nutrients, the term “essential” refers to any nutrient that cannot be synthesized by the body in amounts sufficient for normal growth and to maintain health and that, therefore, must be supplied by the diet. The term “conditionally essential” as applied to nutrients means that the nutrient must be supplied by the diet under conditions when adequate amounts of the precursor compound is unavailable to the body for endogenous synthesis to occur.

“Nutritional supplement” or “supplement” refers to a formulation that contains a nutritionally relevant amount of at least one nutrient. For example, supplements described herein may provide at least one nutrient for a human subject, such as a lactating or pregnant female.

The term “protein equivalent” or “protein equivalent source” includes any protein source, such as soy, egg, whey, or casein, as well as non-protein sources, such as peptides or amino acids. Further, the protein equivalent source can be any used in the art, e.g., nonfat milk, whey protein, casein, soy protein, hydrolyzed protein, peptides, amino acids, and the like. Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate), soy bean proteins, and any combinations thereof. The protein equivalent source can, in some embodiments comprise hydrolyzed protein, including partially hydrolyzed protein and extensively hydrolyzed protein. The protein equivalent source may, in some embodiments, include intact protein. More particularly, the protein source may include a) about 20% to about 80% of the peptide component described herein, and b) about 20% to about 80% of an intact protein, a hydrolyzed protein, or a combination thereof.

The term “protein equivalent source” also encompasses free amino acids. In some embodiments, the amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and mixtures thereof. In some embodiments, the amino acids may be branched chain amino acids. In certain other embodiments, small amino acid peptides may be included as the protein component of the nutritional composition. Such small amino acid peptides may be naturally occurring or synthesized.

The term “essential amino acid” as used herein refers to an amino acid that cannot be synthesized de novo by the organism being considered or that is produced in an insufficient amount, and therefore must be supplied by diet. For example, in some embodiments, where the target subject is a human, an essential amino acid is one that cannot be synthesized de novo by a human.

The term “non-essential amino acid” as used herein refers to an amino acid that can be synthesized by the organism or derived by the organism from essential amino acids. For example, in some embodiments, where the target subject is a human, a non-essential amino acid is one that can be synthesized in the human body or derived in the human body from essential amino acids.

“Probiotic” means a microorganism with low or no pathogenicity that exerts at least one beneficial effect on the health of the host. An example of a probiotic is LGG. In an embodiment, the probiotic(s) may be viable or non-viable. As used herein, the term “viable”, refers to live microorganisms. The term “non-viable” or “non-viable probiotic” means non-living probiotic microorganisms, their cellular components and/or metabolites thereof. Such non-viable probiotics may have been heat-killed or otherwise inactivated, but they retain the ability to favorably influence the health of the host. The probiotics useful in the present disclosure may be naturally-occurring, synthetic or developed through the genetic manipulation of organisms, whether such source is now known or later developed.

The term “non-viable probiotic” means a probiotic wherein the metabolic activity or reproductive ability of the referenced probiotic has been reduced or destroyed. More specifically, “non-viable” or “non-viable probiotic” means non-living probiotic microorganisms, their cellular components and/or metabolites thereof. Such non-viable probiotics may have been heat-killed or otherwise inactivated. The “non-viable probiotic” does, however, still retain, at the cellular level, its cell structure or other structure associated with the cell, for example exopolysaccharide and at least a portion its biological glycol-protein and DNA/RNA structure and thus retains the ability to favorably influence the health of the host. Contrariwise, the term “viable” refers to live microorganisms. As used herein, the term “non-viable” is synonymous with “inactivated”.

The term “cell equivalent” refers to the level of non-viable, non-replicating probiotics equivalent to an equal number of viable cells. The term “non-replicating” is to be understood as the amount of non-replicating microorganisms obtained from the same amount of replicating bacteria (cfu/g), including inactivated probiotics, fragments of DNA, cell wall or cytoplasmic compounds. In other words, the quantity of non-living, non-replicating organisms is expressed in terms of cfu as if all the microorganisms were alive, regardless whether they are dead, non-replicating, inactivated, fragmented etc.

“Prebiotic” means a non-digestible food ingredient that beneficially affects the host by selectively stimulating the growth and/or activity of one or a limited number of beneficial gut bacteria in the digestive tract, selective reduction in gut pathogens, or favorable influence on gut short chain fatty acid profile that can improve the health of the host.

“β-glucan” means all β-glucan, including both β-1,3-glucan and β-1,3;1,6-glucan, as each is a specific type of β-glucan. Moreover, β-1,3;1,6-glucan is a type of β-1,3-glucan. Therefore, the term “β-1,3-glucan” includes β-1,3;1,6-glucan.

All percentages, parts and ratios as used herein are by weight of the total formulation, unless otherwise specified.

The nutritional composition of the present disclosure may be free of substantially free of any optional or selected ingredients described herein. In this context, and unless otherwise specified, the term “substantially free” means that the selected composition may contain less than a functional amount of the optional ingredient, typically less than 0.1% by weight, and also, including zero percent by weight of such optional or selected ingredient.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The compositions and methods of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified in any range. Any reference to a range should be considered as providing support for any subset within that range.

The present disclosure generally relates to pediatric nutritional compositions comprising HMO or precursors thereof. In some embodiments the nutritional compositions include HMO in combination with a prebiotic such as GOS. The combination of GOS and HMO is believed to work together in a complementary and/or synergistic manner by stimulating the growth and activity of beneficial gut microbiota. Gut microbiota are important in normal healthy brain function and development in human infants. Accordingly, the present compositions are believed to promote healthy brain development and function. More particularly, the present compositions, in some embodiments, improve gut microbiota composition and/or activity by increasing proliferation of Bifidobacterium, Lactobacillus and or Allobaculum species.

HMO are believed to correlate with the presence of beneficial infant specific Bifidobacterium species, such as B. longum, B. infantis, B. breve, and B. bifidium in breast fed infants. Accordingly, the HMO used in the present compositions may provide infant formulas that are functionally closer to human milk. Furthermore, the HMO may work synergistically with GOS to further promote the gut-brain axis, thereby providing immediate and lifelong gastrointestinal and neurological benefits to pediatric subjects.

More specifically, the present compositions may modulate a subject's neural development and function, both centrally and peripherally via the enteric nervous system. While not being bound by theory, it is believed that the interactions across the developing gut-brain axis promote neurological development and function in pediatric populations. Additional neurologic benefits may include promoting visual function, sensorimotor development, exploration and manipulation, greater learning and memory, social and emotional development, healthy sleep patterns, and stress reduction.

Accordingly, the present disclosure provides in some embodiments a nutritional composition comprising: (i) a protein source, (ii) a lipid source, (iii) a carbohydrate source, (iv) a human milk oligosaccharide or a precursor thereof, and (v) a prebiotic comprising GOS.

The term “HMO” or “human milk oligosaccharides” refers generally to a number of complex carbohydrates found in human breast milk that can be in either acidic or neutral form. Indeed, HMOs are generally comprised of five monosaccharides, such as glucose, galactose, N-acetylglucosamine, L-fucose, and sialic acid. In certain embodiments, the HMO may be selected from 2′-fucosyllactose, 3′-fucosyllactose, 3′sialyllactose, 6′sialyllactose, lacto-N-biose, lacto-N -neotetraose, lacto-N-tetraose, lacto-N-fucopentaose, lacto-N-fucopentaose I, lacto-N -fucopentaose II, lacto-N-fucopentaose III, lacto-N-fucopentaose V, lacto-N-neofucopentaose, lactodifucotetraose, lacto-N-difucohexaose II, lacto-N-neodifucohexaose II, para-lacto-N -neohexaose, 3′sialyl-3fucosyllactose, sialy-lacto-N-tetraose, L-fucose, N-acetylglucosamine, or any combination thereof.

HMO may be isolated or enriched from milk or produced via microbial fermentation, enzymatic processes, chemical synthesis, or a combination thereof. For example, the HMO disclosed herein may be derived from cow milk, cow colostrum, goat milk, goat colostrum, horse milk, horse colostrum, or any combination thereof. In some embodiments, HMO precursors include sialic acid, fucose, or a combination thereof.

The HMO, in certain embodiments, is present in the compositions in an amount ranging from about 0.005 g/100 kcal to about 1 g/100 kcal. In other embodiments, the HMO may be present in an amount ranging from about 0.01 g/100 kcal to about 1 g/100 kcal, about 0.02 g/100 kcal to about 1 g/100 kcal, about 0.3 g to about 1 g/100 kcal, about 0.1 g/100 kcal to about 0.8 g/100 kcal, or about 0.1 g/100 kcal to about 0.5 g/100 kcal.

In some embodiments, the nutritional composition comprises both a first HMO component and a second HMO component. Indeed, in these embodiments, the first HMO component comprises at least one of the following HMOs: 2′-fucosyllactose, 3′-fucosyllactose, 3′sialyllactose, 6′sialyllactose, lacto-N-biose, lacto-N-neotetraose, lacto-N-tetraose, lacto-N -fucopentaose, lacto-N-fucopentaose I, lacto-N-fucopentaose II, lacto-N-fucopentaose III, lacto -N-fucopentaose V, lacto-N-neofucopentaose, lactodifucotetraose, lacto-N-difucohexaose II, lacto-N-neodifucohexaose II, para-lacto-N-neohexaose, 3′sialyl-3fucosyllactose, sialy-lacto-N -tetraose. Indeed, this first HMO component may be present in an amount of 0.1 mg/mL to about 15 mg/mL. In some embodiments, the first HMO component may be present in an amount of from about 0.5 mg/mL to about 10 mg/mL. In certain embodiments, the first HMO component may be present in an amount of from about 2.0 mg/mL to about 8 mg/mL Still in other embodiments, the first HMO component may be present in an amount of from about 4.0 mg/mL to about 6.0 mg/mL

Indeed, this first HMO component may be present in an amount of 0.015 g/100 kcal to about 2.2 g/100 kcal. In some embodiments, the first HMO component may be present in an amount of from about 0.075 g/100 kcal to about 1.5 g/100 kcal. In certain embodiments, the first HMO component may be present in an amount of from about 0.3 g/100 kcal to about 1.2 g/100 kcal Still in other embodiments, the first HMO component may be present in an amount of from about 0.6 g/100 kcal to about 0.9 g/100 kcal.

In some embodiment, the nutritional composition may include a second HMO component including an HMO or precursor thereof selected from N-acetylglucosamine and/or L-fucose. N-acetylglucosamine is a building block of HMO and is also a component of lacto-N -tetraose and lacto-N-neotetraose. L-fucose is normally found in the mammalian gut and may provide a protective role against inflammation and infection. Furthermore, L-fucose may function as a food source or adhesion site in the gut for beneficial gut bacteria and, at the same time, suppress the virulence of pathogens. Additionally, L-fucose can also modulate presynaptic and postsynaptic mechanism in the central nervous system, indeed, it was shown that administration of L-fucose improves memory consolidation by prolonging hippocampal long term potentiation.

In certain embodiments, the second HMO component is present in the nutritional composition in an amount of from about 0.01 mg/mL to about 8 mg/mL. In some embodiments, the second HMO component is present in an amount of from about 0.1 mg/mL to about 5.0 mg/mL. Still in certain embodiments, the second HMO component is present in an amount of from about 1.5 mg/mL to about 4 mg/mL.

In certain embodiments, the second HMO component is present in the nutritional composition in an amount of from about 0.0015 g/100 kcal to about 1.2 g/100 kcal. In some embodiments, the second HMO component is present in an amount of from about 0.015 g/100 kcal to about 0.75 g/100 kcal. Still in certain embodiments, the second HMO component is present in an amount of from about 0.22 g/100 kcal to about 0.6 g/100 kcal.

Indeed, many current infant formulas or pediatric nutritional products are not supplemented with acidic oligosaccharides, such as sialyllactose, or other types of HMO as these components have historically been of limited availability. Further, formulating a shelf-stable nutritional composition capable of providing and effective amount of HMO has been a hurdle for infant formula manufacturers. However, as provided herein, the HMO are formulated in the nutritional compositions in an amount of from about 0.005 g to about 1.0 g per 100 kcal, which ensures that an effective amount is administered to the infant or pediatric subject. Furthermore, formulating a nutritional composition including this amount of HMO based on a 100 kcal serving, further ensures that the product remains shelf-stable during storage and the bioactivity of the HMO is not lost during storage. Accordingly, in some embodiments herein, the nutritional composition formulated with the specific amount of HMO per a 100 kcal serving ensures that the beneficial health benefits disclosed herein are provided to the target subject.

Furthermore, in some embodiments the HMO provided are fucosylated and/or sialylated. Indeed, the present disclosure provides for a nutritional composition comprising human milk oligosaccharides, wherein: (a) about 60-80% of the HMO are sialylated, about 0-20% are fucosylated, and about 10-30% are neither sialylated or fucosylated; (b) about 20-40% of the HMO are sialylated, about 40-60% are fucosylated, and about 10-30% are neither sialylated or fucosylated; or (c) about 10-30% of the HMO are sialylated, about 10-30% are fucosylated, and about 50-70% are neither sialylated or fucosylated.

In certain embodiments, (a) about 70% of the HMO are sialylated, about 10% are fucosylated, and about 20% are neither sialylated or fucosylated; (b) about 30% of the HMO are sialylated, about 50% are fucosylated, and about 20% are neither sialylated or fucosylated; or (c) about 20% of the HMO are sialylated, about 20% are fucosylated, and about 60% are neither sialylated or fucosylated.

In some embodiments the nutritional compositions include from about 0.01 g/100 kcal to about 0.8 g/100 kcal of sialylated HMO. In other embodiments, the nutritional compositions include from about 0.03 g/100 kcal to about 0.6 g/100 kcal of sialylated HMO. Still in some embodiments, then nutritional compositions include from about 0.04 g/100 kcal to about 0.8 g/100 kcal of sialylated HMO. Still in other embodiments, the nutritional compositions include from about 0.05 g/100 kcal to about 0.6 g/100 kcal of sialylated HMO.

In some embodiments, the nutritional compositions include from about 0.01 g/100 kcal to about 0.2 g/100 kcal of fucosylated HMO. In some embodiments, the nutritional compositions include from about 0.02 g/100 kcal to about 0.2 g/100 kcal of fucosylated HMO. In some embodiments, the nutritional compositions include from about 0.05 g/100 kcal to about 0.1 g/100 kcal of fucosylated HMO.

In some embodiments, the nutritional compositions include from about 0.01 g/100 kcal to about 0.5 g/100 kcal of HMO that are neither sialyated nor fucosylated. IN certain embodiments, the nutritional compositions include from about 0.025 g/100 kcal to about 0.5 g/100 kcal of HMO that are neither sialylated nor fucosylated. In other embodiments, the nutritional compositions contain from about 0.25 g/100 kcal to about 0.7 g/100 kcal of HMO that are neither sialylated nor fucosylated. Indeed, in certain embodiments, the majority of the HMO included in the nutritional compositions are neither sialylated nor fucosylated.

In some embodiments, the nutritional composition may be formulated to include a certain weight percentage of HMO based on the total amount of carbohydrates present in the nutritional compositions. Accordingly, in some embodiments the nutritional composition may include from about 0.1 wt % to about 25 wt % HMO based on the total weight of carbohydrates in the nutritional composition. In some embodiments, the nutritional composition includes from about 0.5wt % to about 25 wt % HMO based on the total weight of carbohydrates in the nutritional composition. In some embodiments, the nutritional composition includes from about 1 wt % to about 25 wt % HMO based on the total weight of carbohydrates in the nutritional composition. In some embodiments, the nutritional composition includes from about 2 wt % to about 20 wt % HMO based on the total weight of the carbohydrates in the nutritional composition. Still in some embodiments, the nutritional composition includes from about 5 wt % to about 15 wt % HMO based on the total weight of the carbohydrates in the nutritional composition. In some embodiments, the nutritional composition includes from about 8 wt % to about 12 wt % HMO based on the total weight of the carbohydrates in the nutritional composition. Still, in certain embodiments, the nutritional composition is formulated to include from about 0.1 wt % to about 5 wt % of HMO based on the total weight of the carbohydrates in the nutritional composition.

The nutritional composition may also contain one or more prebiotics (also referred to as a prebiotic source) in certain embodiments. Prebiotics can stimulate the growth and/or activity of ingested probiotic microorganisms, selectively reduce pathogens found in the gut, and favorably influence the short chain fatty acid profile of the gut. Such prebiotics may be naturally-occurring, synthetic, or developed through the genetic manipulation of organisms and/or plants, whether such new source is now known or developed later. Prebiotics useful in the present disclosure may include oligosaccharides, polysaccharides, and other prebiotics that contain fructose, xylose, soya, galactose, glucose and mannose.

More specifically, prebiotics useful in the present disclosure may include polydextrose, polydextrose powder, lactulose, lactosucrose, raffinose, gluco-oligosaccharide, inulin, fructo -oligosaccharide, isomalto-oligosaccharide, soybean oligosaccharides, lactosucrose, xylo -oligosaccharide, chito-oligosaccharide, manno-oligosaccharide, aribino-oligosaccharide, siallyl -oligosaccharide, fuco-oligosaccharide, galacto-oligosaccharide, and gentio-oligosaccharides. In some embodiments, the total amount of prebiotics present in the nutritional composition may be from about 0.1 g/100 kcal to about 1.5 g/100 kcal. In certain embodiments, the total amount of prebiotics present in the nutritional composition may be from about 0.3 g/100 kcal to about 1.0 g/100 kcal. Moreover, the nutritional composition may comprise a prebiotic component comprising polydextrose (“PDX”) and/or galacto-oligosaccharide (“GOS”). In some embodiments, the prebiotic component comprises at least 20% GOS, PDX or a mixture thereof.

In some embodiments, the HMO component may be included in combination with GOS and PDX. In these embodiments, the nutritional composition may comprise from about 0.1 g/100 kcal to about 5 g/100 kcal of prebiotics, including GOS, PDX, and HMO. Still in certain embodiments, the nutritional composition may include from about 0.1 g/100 kcal to about 4 g/100 kcal of prebiotics, including GOS, PDX, and HMO.

The disclosed nutritional compositions comprise a source of prebiotics, specifically GOS and/or PDX, in addition to the HMO component. In some embodiments, at least 20% of the prebiotic component comprises GOS. In other embodiments, the prebiotic component comprises both GOS and PDX. The GOS and PDX may be present in a ratio of about 1:9 to about 9:1 by weight. In other embodiments, the GOS and PDX are present in a ratio of about 1:4 to 4:1, or about 1:1. In another embodiment, the ratio of PDX:GOS can be between about 5:1 and 1:5. In yet another embodiment, the ratio of PDX:GOS can be between about 1:3 and 3:1. In a particular embodiment, the ratio of PDX to GOS can be about 5:5. In another particular embodiment, the ratio of PDX to GOS can be about 8:2.

In some embodiments, the amount of GOS in the nutritional composition may be from about 0.1 g/100 kcal to about 1.0 g/100 kcal. In another embodiment, the amount of GOS in the nutritional composition may be from about 0.1 g/100 kcal to about 0.5 g/100 kcal. The amount of PDX in the nutritional composition may, in some embodiments, be within the range of from about 0.1 g/100 kcal to about 0.5 g/100 kcal. In other embodiments, the amount of PDX may be about 0.3 g/100 kcal.

In a particular embodiment, GOS and PDX are supplemented into the nutritional composition in a total amount of about at least about 0.2 g/100 kcal and can be about 0.2 g/100 kcal to about 1.5 g/100 kcal. In some embodiments, the nutritional composition may comprise GOS and PDX in a total amount of from about 0.6 to about 0.8 g/100 kcal.

In a particular embodiment, GOS and PDX are supplemented into the nutritional composition in a total amount of about at least about 1.5 g/L and can be about 1.5 g/L to about 10 g/L. In some embodiments, the nutritional composition may comprise GOS and PDX in a total amount of from about 4.0 to about 5.5 g/L.

In some embodiments, the nutritional composition may include prebiotics in addition to GOS and PDX. In some embodiments, additional prebiotics useful in the present disclosure may include: lactulose, lactosucrose, raffinose, gluco-oligosaccharide, inulin, fructo -oligosaccharide, isomalto-oligosaccharide, soybean oligosaccharides, lactosucrose, xylo -oligosaccharide, chito-oligosaccharide, manno-oligosaccharide, aribino-oligosaccharide, siallyl -oligosaccharide, fuco-oligosaccharide, and gentio-oligosaccharides. In embodiments where GOS and PDX are not included at the upper limit of their respective concentration range, additional prebiotics may be included up to the upper limit concentration specified.

In some embodiments, the nutritional composition comprises a first HMO component a second HMO component, and a prebiotic component that includes GOS. In this embodiment, the prebiotic component including GOS may be present in an amount of from about 0.1 mg/mL to about 10 mg/mL. In some embodiments, the prebiotic component including GOS may be present in an amount of from about 1 mg/mL to about 6 mg/mL. In some embodiments, the prebiotic component including GOS may be present in an amount of from about 2.0 mg/mL to about 4.0 mg/mL.

In some embodiments, the nutritional composition may contain one or more probiotics. Any probiotic known in the art may be acceptable in this embodiment. In a particular embodiment, the probiotic may be selected from any Lactobacillus species, Lactobacillus rhamnosus GG (ATCC number 53103 “LGG”), Bifidobacterium species, Bifidobacterium longum BB536 (BL999, ATCC: BAA-999), Bifidobacterium longum AH1206 (NCIMB: 41382), Bifidobacterium breve AH1205 (NCIMB: 41387), Bifidobacterium infantis 35624 (NCIMB: 41003), and Bifidobacterium animalis subsp. lactis BB-12 (DSM No. 10140) or any combination thereof.

In some embodiments, the nutritional composition includes a probiotic, and more particularly, LGG, in an amount of from about 1×10⁴ cfu/100 kcal to about 1.5×10¹⁰ cfu/100 kcal. In other embodiments, the nutritional composition comprises LGG in an amount of from about 1×10⁶ cfu/100 kcal to about 1×10⁹ cfu/100 kcal. Still, in certain embodiments, the nutritional composition may include LGG in an amount of from about 1×10⁷ cfu/100 kcal to about 1×10⁸ cfu/100 kcal. In some embodiments, where LGG is not included at the upper limit of the concentration range, additional probiotics may be included up to the upper limit concentration specified.

In an embodiment, the probiotic(s) may be viable or non-viable. As used herein, the term “viable”, refers to live microorganisms.

While, probiotics may be helpful in pediatric patients, the administration of viable bacteria to pediatric subjects, and particularly preterm infants, with impaired intestinal defenses and immature gut barrier function may not be feasible due to the risk of bacteremia. Therefore, there is a need for compositions that can provide the benefits of probiotics without introducing viable bacteria into the intestinal tract of pediatric subjects

While not wishing to be bound by theory, it is believed that a culture supernatant from batch cultivation of a probiotic, and in particular embodiments, LGG, provides beneficial gastrointestinal benefits. It is further believed that the beneficial effects on gut barrier function can be attributed to the mixture of components (including proteinaceous materials, and possibly including (exo)polysaccharide materials) that are released into the culture medium at a late stage of the exponential (or “log”) phase of batch cultivation of LGG. The composition will be hereinafter referred to as “culture supernatant.”

Accordingly, in some embodiments, the nutritional composition includes a culture supernatant from a late-exponential growth phase of a probiotic batch-cultivation process. Without wishing to be bound by theory, it is believed that the activity of the culture supernatant can be attributed to the mixture of components (including proteinaceous materials, and possibly including (exo)polysaccharide materials) as found released into the culture medium at a late stage of the exponential (or “log”) phase of batch cultivation of the probiotic. The term “culture supernatant” as used herein, includes the mixture of components found in the culture medium. The stages recognized in batch cultivation of bacteria are known to the skilled person. These are the “lag,” the “log” (“logarithmic” or “exponential”), the “stationary” and the “death” (or “logarithmic decline”) phases. In all phases during which live bacteria are present, the bacteria metabolize nutrients from the media, and secrete (exert, release) materials into the culture medium. The composition of the secreted material at a given point in time of the growth stages is not generally predictable.

In some embodiments, the probiotic functionality in the nutritional composition of the present disclosure is provided by including a culture supernatant from a late-exponential growth phase of a probiotic batch-cultivation process, as disclosed in international published application no. WO 2013/142403, which is hereby incorporated by reference in its entirety. The stages recognized in batch cultivation of bacteria are known to the skilled person. These are the “lag,” the “log” (“logarithmic” or “exponential”), the “stationary” and the “death” (or “logarithmic decline”) phases. In all phases during which live bacteria are present, the bacteria metabolize nutrients from the media, and secrete (exert, release) materials into the culture medium. The composition of the secreted material at a given point in time of the growth stages is not generally predictable.

In an embodiment, a culture supernatant is obtainable by a process comprising the steps of (a) subjecting a probiotic such as LGG to cultivation in a suitable culture medium using a batch process; (b) harvesting the culture supernatant at a late exponential growth phase of the cultivation step, which phase is defined with reference to the second half of the time between the lag phase and the stationary phase of the batch-cultivation process; (c) optionally removing low molecular weight constituents from the supernatant so as to retain molecular weight constituents above 5-6 kiloDaltons (kDa); (d) removing liquid contents from the culture supernatant so as to obtain the composition.

The culture supernatant may comprise secreted materials that are harvested from a late exponential phase. The late exponential phase occurs in time after the mid exponential phase (which is halftime of the duration of the exponential phase, hence the reference to the late exponential phase as being the second half of the time between the lag phase and the stationary phase). In particular, the term “late exponential phase” is used herein with reference to the latter quarter portion of the time between the lag phase and the stationary phase of the LGG batch-cultivation process. In some embodiments, the culture supernatant is harvested at a point in time of 75% to 85% of the duration of the exponential phase, and may be harvested at about 5/6 of the time elapsed in the exponential phase.

In an embodiment, a culture supernatant is obtainable by a process comprising the steps of (a) subjecting a probiotic such as LGG to cultivation in a suitable culture medium using a batch process; (b) harvesting the culture supernatant at a late exponential growth phase of the cultivation step, which phase is defined with reference to the second half of the time between the lag phase and the stationary phase of the batch-cultivation process; (c) optionally removing low molecular weight constituents from the supernatant so as to retain molecular weight constituents above 5-6 kiloDaltons (kDa); (d) removing liquid contents from the culture supernatant so as to obtain the composition.

The culture supernatant is believed to contain a mixture of amino acids, oligo- and polypeptides, and proteins, of various molecular weights. The composition is further believed to contain polysaccharide structures and/or nucleotides.

In some embodiments, the culture supernatant of the present disclosure excludes low molecular weight components, generally below 6 kDa, or even below 5 kDa. In these and other embodiments, the culture supernatant does not include lactic acid and/or lactate salts. These lower molecular weight components can be removed, for example, by filtration or column chromatography.

The culture supernatant of the present disclosure can be formulated in various ways for administration to pediatric subjects. For example, the culture supernatant can be used as such, e.g. incorporated into capsules for oral administration, or in a liquid nutritional composition such as a drink, or it can be processed before further use. Such processing generally involves separating the compounds from the generally liquid continuous phase of the supernatant. This preferably is done by a drying method, such as spray-drying or freeze-drying (lyophilization). Spray-drying is preferred. In a preferred embodiment of the spray-drying method, a carrier material will be added before spray-drying, e.g., maltodextrin DE29.

The LGG culture supernatant of the present disclosure, whether added in a separate dosage form or via a nutritional product, will generally be administered in an amount effective in promoting gut regeneration, promoting gut maturation and/or protecting gut barrier function. The effective amount is preferably equivalent to 1×10⁴ to about 1×10¹² cell equivalents of live probiotic bacteria per kg body weight per day, and more preferably 10⁸-10⁹ cell equivalents per kg body weight per day. In other embodiments, the amount of cell equivalents may vary from about 1×10⁴ to about 1.5×10¹⁰ cell equivalents of probiotic(s) per 100 Kcal. In some embodiments the amount of probiotic cell equivalents may be from about 1×10⁶ to about 1×10⁹ cell equivalents of probiotic(s) per 100 Kcal nutritional composition. In certain other embodiments the amount of probiotic cell equivalents may vary from about 1×10⁷ to about 1×10⁸ cell equivalents of probiotic(s) per 100 Kcal of nutritional composition.

In some embodiments, a soluble mediator preparation is prepared from the culture supernatant as described below. Furthermore, preparation of an LGG soluble mediator preparation is described in US 20130251829 and US 20110217402, each of which is incorporated by reference in its entirety. The stages recognized in batch cultivation of bacteria are known to the skilled person. These are the “lag,” the “log” (“logarithmic” or “exponential”), the “stationary” and the “death” (or “logarithmic decline”) phases. In all phases during which live bacteria are present, the bacteria metabolize nutrients from the media, and secrete (exert, release) materials into the culture medium. The composition of the secreted material at a given point in time of the growth stages is not generally predictable.

In certain embodiments, the soluble mediator preparation is obtainable by a process comprising the steps of (a) subjecting a probiotic such as LGG to cultivation in a suitable culture medium using a batch process; (b) harvesting a culture supernatant at a late exponential growth phase of the cultivation step, which phase is defined with reference to the second half of the time between the lag phase and the stationary phase of the batch-cultivation process; (c) optionally removing low molecular weight constituents from the supernatant so as to retain molecular weight constituents above 5-6 kiloDaltons (kDa); (d) removal of any remaining cells using 0.22 μm sterile filtration to provide the soluble mediator preparation; (e) removing liquid contents from the soluble mediator preparation so as to obtain the composition.

In certain embodiments, secreted materials are harvested from a late exponential phase. The late exponential phase occurs in time after the mid exponential phase (which is halftime of the duration of the exponential phase, hence the reference to the late exponential phase as being the second half of the time between the lag phase and the stationary phase). In particular, the term “late exponential phase” is used herein with reference to the latter quarter portion of the time between the lag phase and the stationary phase of the LGG batch-cultivation process. In a preferred embodiment of the present disclosure and embodiments thereof, harvesting of the culture supernatant is at a point in time of 75% to 85% of the duration of the exponential phase, and most preferably is at about 5/6 of the time elapsed in the exponential phase.

The term “cultivation” or “culturing” refers to the propagation of micro-organisms, in this case LGG, on or in a suitable medium. Such a culture medium can be of a variety of kinds, and is particularly a liquid broth, as customary in the art. A preferred broth, e.g., is MRS broth as generally used for the cultivation of lactobacilli. MRS broth generally comprises polysorbate, acetate, magnesium and manganese, which are known to act as special growth factors for lactobacilli, as well as a rich nutrient base. A typical composition comprises (amounts in g/liter): peptone from casein 10.0; meat extract 8.0; yeast extract 4.0; D(+)-glucose 20.0; dipotassium hydrogen phosphate 2.0; Tween® 80 1.0; triammonium citrate 2.0; sodium acetate 5.0; magnesium sulphate 0.2; manganese sulphate 0.04.

In certain embodiments, the soluble mediator preparation is incorporated into an infant formula or other nutritional composition. The harvesting of secreted bacterial products brings about a problem that the culture media cannot easily be deprived of undesired components. This specifically relates to nutritional products for relatively vulnerable subjects, such as infant formula or clinical nutrition. This problem is not incurred if specific components from a culture supernatant are first isolated, purified, and then applied in a nutritional product. However, it is desired to make use of a more complete culture supernatant. This would serve to provide a soluble mediator composition better reflecting the natural action of the probiotic (e.g. LGG).

Accordingly, it is desired to ensure that the composition harvested from LGG cultivation does not contain components (as may present in the culture medium) that are not desired, or generally accepted, in such formula. With reference to polysorbate regularly present in MRS broth, media for the culturing of bacteria may include an emulsifying non-ionic surfactant, e.g. on the basis of polyethoxylated sorbitan and oleic acid (typically available as Tween® polysorbates, such as Tween® 80). Whilst these surfactants are frequently found in food products, e.g. ice cream, and are generally recognized as safe, they are not in all jurisdictions considered desirable, or even acceptable for use in nutritional products for relatively vulnerable subjects, such as infant formula or clinical nutrition.

Therefore, in some embodiments, a preferred culture medium of the disclosure is devoid of polysorbates such as Tween 80. In a preferred embodiment of the disclosure and/or embodiments thereof the culture medium may comprise an oily ingredient selected from the group consisting of oleic acid, linseed oil, olive oil, rape seed oil, sunflower oil and mixtures thereof. It will be understood that the full benefit of the oily ingredient is attained if the presence of a polysorbate surfactant is essentially or entirely avoided.

More particularly, in certain embodiments, an MRS medium is devoid of polysorbates. Also preferably medium comprises, in addition to one or more of the foregoing oils, peptone (typically 0-10 g/L, especially 0.1-10 g/L), meat extract (typically 0-8 g/L, especially 0.1-8 g/L), yeast extract (typically 4-50 g/L), D(+) glucose (typically 20-70 g/L), dipotassium hydrogen phosphate (typically 2-4 g/L), sodium acetate trihydrate (typically 4-5 g/L), triammonium citrate (typically 2-4 g/L), magnesium sulfphate heptahydrate (typically 0.2-0.4 g/L) and/or manganous sulphate tetrahydrate (typically 0.05-0.08 g/L).

The culturing is generally performed at a temperature of 20° C. to 45° C., more particularly at 35° C. to 40° C., and more particularly at 37° C. In some embodiments, the culture has a neutral pH, such as a pH of between pH 5 and pH 7, preferably pH 6.

In some embodiments, the time point during cultivation for harvesting the culture supernatant, i.e., in the aforementioned late exponential phase, can be determined, e.g. based on the OD600 nm and glucose concentration. OD600 refers to the optical density at 600 nm, which is a known density measurement that directly correlates with the bacterial concentration in the culture medium.

The culture supernatant can be harvested by any known technique for the separation of culture supernatant from a bacterial culture. Such techniques are known in the art and include, e.g., centrifugation, filtration, sedimentation, and the like. In some embodiments, LGG cells are removed from the culture supernatant using 0.22 [symbol] m sterile filtration in order to produce the soluble mediator preparation. The probiotic soluble mediator preparation thus obtained may be used immediately, or be stored for future use. In the latter case, the probiotic soluble mediator preparation will generally be refrigerated, frozen or lyophilized. The probiotic soluble mediator preparation may be concentrated or diluted, as desired.

The soluble mediator preparation is believed to contain a mixture of amino acids, oligo- and polypeptides, and proteins, of various molecular weights. The composition is further believed to contain polysaccharide structures and/or nucleotides.

In some embodiments, the soluble mediator preparation of the present disclosure excludes lower molecular weight components, generally below 6 kDa, or even below 5 kDa. In these and other embodiments, the soluble mediator preparation does not include lactic acid and/or lactate salts. These lower molecular weight components can be removed, for example, by filtration or column chromatography. In some embodiments, the culture supernatant is subjected to ultrafiltration with a 5 kDa membrane in order to retain constituents over 5 kDa. In other embodiments, the culture supernatant is desalted using column chromatography to retain constituents over 6 kDa.

The soluble mediator preparation of the present disclosure can be formulated in various ways for administration to pediatric subjects. For example, the soluble mediator preparation can be used as such, e.g. incorporated into capsules for oral administration, or in a liquid nutritional composition such as a drink, or it can be processed before further use. Such processing generally involves separating the compounds from the generally liquid continuous phase of the supernatant. This preferably is done by a drying method, such as spray-drying or freeze-drying (lyophilization). In a preferred embodiment of the spray-drying method, a carrier material will be added before spray-drying, e.g., maltodextrin DE29.

Probiotic bacteria soluble mediator preparations, such as the LGG soluble mediator preparation disclosed herein, advantageously possess gut barrier enhancing activity by promoting gut barrier regeneration, gut barrier maturation and/or adaptation, gut barrier resistance and/or gut barrier function. The present LGG soluble mediator preparation may accordingly be particularly useful in treating subjects, particularly pediatric subjects, with impaired gut barrier function, such as short bowel syndrome or NEC. The soluble mediator preparation may be particularly useful for infants and premature infants having impaired gut barrier function and/or short bowel syndrome.

Probiotic bacteria soluble mediator preparation, such as the LGG soluble mediator preparation of the present disclosure, also advantageously reduce visceral pain sensitivity in subjects, particularly pediatric subjects experiencing gastrointestinal pain, food intolerance, allergic or non-allergic inflammation, colic, IBS, and infections.

The nutritional composition of the disclosure may contain a source of long chain polyunsaturated fatty acid (LCPUFA) that comprises docosahexaenoic acid (DHA). Non-limiting examples of LCPUFAs include, but are not limited to, DHA, ARA, linoleic (18:2 n-6), γ-linolenic (18:3 n-6), dihomo-γ-linolenic (20:3 n-6) acids in the n-6 pathway, α-linolenic (18:3 n-3), stearidonic (18:4 n-3), eicosatetraenoic (20:4 n-3), eicosapentaenoic (20:5 n-3), and docosapentaenoic (22:6 n-3). In an embodiment, especially if the nutritional composition is an infant formula, the nutritional composition is supplemented with both DHA and ARA. In this embodiment, the weight ratio of ARA:DHA may be between about 1:3 and about 9:1. In a particular embodiment, the ratio of ARA:DHA is from about 1:2 to about 4:1.

If included, the source of DHA and/or ARA may be any source known in the art such as marine oil, fish oil, single cell oil, egg yolk lipid, and brain lipid. In some embodiments, the DHA and ARA are sourced from single cell Martek oils, DHASCO® and ARASCO®, or variations thereof. The DHA and ARA can be in natural form, provided that the remainder of the LCPUFA source does not result in any substantial deleterious effect on the subject. Alternatively, the DHA and ARA can be used in refined form.

In an embodiment, sources of DHA and ARA are single cell oils as taught in U.S. Pat. Nos. 5,374,657; 5,550,156; and 5,397,591, the disclosures of which are incorporated herein in their entirety by reference. Nevertheless, the present disclosure is not limited to only such oils.

In some embodiments the nutritional composition may also include a source of LCPUFAs. In one embodiment the amount of LCPUFA in the nutritional composition is advantageously at least about 5 mg/100 Kcal, and may vary from about 5 mg/100 Kcal to about 100 mg/100 Kcal, more preferably from about 10 mg/100 Kcal to about 50 mg/100 Kcal.

In some embodiments, the LCPUFA included in the nutritional composition may comprise DHA. In one embodiment the amount of DHA in the nutritional composition is advantageously at least about 17 mg/100 Kcal, and may vary from about 5 mg/100 Kcal to about 75 mg/100 Kcal, more preferably from about 10 mg/100 Kcal to about 50 mg/100 Kcal.

The nutritional composition may also comprise a source of β-glucan. Glucans are polysaccharides, specifically polymers of glucose, which are naturally occurring and may be found in cell walls of bacteria, yeast, fungi, and plants. Beta glucans (β-glucans) are themselves a diverse subset of glucose polymers, which are made up of chains of glucose monomers linked together via beta-type glycosidic bonds to form complex carbohydrates.

β-1,3-glucans are carbohydrate polymers purified from, for example, yeast, mushroom, bacteria, algae, or cereals. The chemical structure of β-1,3-glucan depends on the source of the β-1,3-glucan. Moreover, various physiochemical parameters, such as solubility, primary structure, molecular weight, and branching, play a role in biological activities of β-1,3-glucans.

β-1,3-glucans are naturally occurring polysaccharides, with or without β-1,6-glucose side chains that are found in the cell walls of a variety of plants, yeasts, fungi and bacteria. β-1,3;1,6-glucans are those containing glucose units with (1,3) links having side chains attached at the (1,6) position(s). β-1,3;1,6 glucans are a heterogeneous group of glucose polymers that share structural commonalities, including a backbone of straight chain glucose units linked by a β-1,3 bond with β-1,6-linked glucose branches extending from this backbone. While this is the basic structure for the presently described class of β-glucans, some variations may exist. For example, certain yeast β-glucans have additional regions of β(1,3) branching extending from the β(1,6) branches, which add further complexity to their respective structures.

β-glucans derived from baker's yeast, Saccharomyces cerevisiae, are made up of chains of D-glucose molecules connected at the 1 and 3 positions, having side chains of glucose attached at the 1 and 6 positions. Yeast-derived β-glucan is an insoluble, fiber-like, complex sugar having the general structure of a linear chain of glucose units with a β-1,3 backbone interspersed with β-1,6 side chains that are generally 6-8 glucose units in length. More specifically, β-glucan derived from baker's yeast is poly-(1,6)-β-D-glucopyranosyl-(1,3)-β-D -glucopyranose.

Furthermore, β-glucans are well tolerated and do not produce or cause excess gas, abdominal distension, bloating or diarrhea in pediatric subjects. Addition of (β-glucan to a nutritional composition for a pediatric subject, such as an infant formula, a growing-up milk or another children's nutritional product, will improve the subject's immune response by increasing resistance against invading pathogens and therefore maintaining or improving overall health.

In some embodiments, the amount of β-glucan in the nutritional composition is between about 3 mg and about 17 mg per 100 Kcal. In another embodiment the amount of β-glucan is between about 6 mg and about 17 mg per 100 Kcal.

In a particular embodiment, a nutritional composition comprises per 100 kcal: (i) between about 1 g and about 7 g of a protein source, (ii) between about 1 g and about 10 g of a lipid source, (iii) between about 6 g and about 22 g of a carbohydrate source, (iv) between about 0.05 g and about 1 g of a human milk oligosaccharide, (v) between about 0.1 g and 1.0 g of a galacto-oligosaccharide, and (vi) between about 1×10⁵ cfu/100 kcals to about 1.5×10⁹ cfu/100 kcals of Lactobacillus rhamnosus GG or about 1×10⁵ cell equivalents per 100 kcals to about 1.5×10⁹ cell equivalents per 100 kcals of dry composition of Lactobacillus rhamnosus GG. In some embodiments, the nutritional composition comprises the culture supernatant from about 0.015 g per 100 kcal to about 1.5 g per 100 kcal.

The present disclosure also provides a method for promoting the growth of beneficial microbiota in the gastrointestinal tract of pediatric subject in need thereof comprising administering to the subject an effective amount of any of the nutritional compositions described herein, for example a nutritional composition comprising a first HMO component containing at least one of 3′-sialyllactose, 6′-sialyllactose, lacto-N-biose, lacto-N-neotetratose, lacto-N-tetraose, and combinations thereof; a second HMO component including L-fucose and/or N-acetylglucosamine; and GOS. More particularly, the present disclosure provides a method for promoting the growth of beneficial microbiota in the gastrointestinal tract of pediatric subject in need thereof comprising administering to the subject an effective amount of a nutritional composition comprising: (i) a protein source, (ii) a lipid source, (iii) a carbohydrate source, (iv) a first HMO containing at least one of 3′-sialyllactose, 6′-sialyllactose, lacto-N-biose, lacto-N-neotetratose, lacto-N-tetraose, and combinations thereof, (v) a second HMO component including L-fucose and/or N-acetylglucosamine, and (vi) GOS.

The disclosed nutritional composition(s) may be provided in any form known in the art, such as a powder, a gel, a suspension, a paste, a solid, a liquid, a liquid concentrate, a reconstitutable powdered milk substitute or a ready-to-use product. The nutritional composition may, in certain embodiments, comprise a nutritional supplement, children's nutritional product, infant formula, human milk fortifier, growing-up milk or any other nutritional composition designed for a pediatric subject. Nutritional compositions of the present disclosure include, for example, orally-ingestible, health-promoting substances including, for example, foods, beverages, tablets, capsules and powders. Moreover, the nutritional composition of the present disclosure may be standardized to a specific caloric content, it may be provided as a ready-to-use product, or it may be provided in a concentrated form. In some embodiments, the nutritional composition is in powder form with a particle size in the range of 5 μm to 1500 μm, more preferably in the range of 10 μm to 1000 μm, and even more preferably in the range of 50 μm to 300 μm.

In some embodiments, the nutritional composition is an infant formula suitable for infants ranging in age from 0 to 12 months, from 0 to 3 months, 0 to 6 months or 6 to 12 months. In other embodiments, the disclosure provides a fortified milk-based growing-up milk designed for children ages 1-3 years and/or 4-6 years, wherein the growing-up milk supports growth and development and life-long health.

As noted, the nutritional composition(s) of the disclosure may comprise a protein source. The protein source can be any used in the art, e.g., nonfat milk, whey protein, casein, soy protein, hydrolyzed protein, amino acids, and the like. Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate) and any combinations thereof.

In one embodiment, the proteins of the nutritional composition are provided as intact proteins. In other embodiments, the proteins are provided as a combination of both intact proteins and partially hydrolyzed proteins, with a degree of hydrolysis of between about 4% and 10%. In certain other embodiments, the proteins are more completely hydrolyzed. In still other embodiments, the protein source comprises amino acids as a protein equivalent. In yet another embodiment, the protein source may be supplemented with glutamine-containing peptides.

In a particular embodiment of the nutritional composition, the whey:casein ratio of the protein source is similar to that found in human breast milk. In an embodiment, the protein source comprises from about 40% to about 90% whey protein and from about 10% to about 60% casein.

In some embodiments, the nutritional composition comprises between about 1 g and about 7 g of a protein source per 100 kcal. In other embodiments, the nutritional composition comprises between about 3.5 g and about 4.5 g of protein per 100 kcal.

In some embodiments, the protein equivalent source comprises a hydrolyzed protein, which includes partially hydrolyzed protein and extensively hydrolyzed protein, such as casein. In some embodiments, the protein equivalent source comprises a hydrolyzed protein including peptides having a molar mass distribution of greater than 500 Daltons. In some embodiments, the hydrolyzed protein comprises peptides having a molar mass distribution in the range of from about 500 Daltons to about 1,500 Daltons. Still, in some embodiments the hydrolyzed protein may comprise peptides having a molar mass distribution range of from about 500 Daltons to about 2,000 Daltons.

In some embodiments, the protein equivalent source may comprise the peptide component, intact protein, hydrolyzed protein, including partially hydrolyzed protein and/or extensively hydrolyzed protein, and combinations thereof. In some embodiments, 20% to 80% of the protein equivalent source comprises the peptide component disclosed herein. In some embodiments, 30% to 60% of the protein equivalent source comprises the peptide component disclosed herein. In still other embodiments, 40% to 50% of the protein equivalent source comprises the peptide component.

In some embodiments, 20% to 80% of the protein equivalent source comprises intact protein, partially hydrolyzed protein, extensively hydrolyzed protein, or combinations thereof. In some embodiments, 40% to 70% of the protein equivalent source comprises intact proteins, partially hydrolyzed proteins, extensively hydrolyzed protein, or a combination thereof. In still further embodiments, 50% to 60% of the protein equivalent source may comprise intact proteins, partially hydrolyzed protein, extensively hydrolyzed protein, or a combination thereof.

In some embodiments the protein equivalent source comprises partially hydrolyzed protein having a degree of hydrolysis of less than 40%. In still other embodiments, the protein equivalent source may comprise partially hydrolyzed protein having a degree of hydrolysis of less than 25%, or less than 15%.

In some embodiments, the nutritional composition comprises between about 1 g and about 7 g of a protein equivalent source per 100 Kcal. In other embodiments, the nutritional composition comprises between about 3.5 g and about 4.5 g of protein equivalent source per 100 Kcal.

In certain embodiments, the protein equivalent source comprises amino acids and is substantially free of whole, intact protein. Further in certain embodiments, the protein equivalent source comprises amino acids and is substantially free of peptides. In certain embodiments, the protein equivalent source includes from about 10% to about 90% w/w of essential amino acids based on the total amino acids included in the protein equivalent source. In certain embodiments, the protein equivalent source includes from about 25% to about 75% w/w of essential amino acids based on the total amino acids included in the protein equivalent source. In some embodiments, the protein equivalent source includes from about 40% to about 60% of essential amino acids based on the total amino acids included in the protein equivalent source.

In some embodiments, the protein equivalent source includes non-essential amino acids. In certain embodiments, the protein equivalent source includes from about 10% to about 90% w/w of non-essential amino acids based on the total amino acids included in the protein equivalent source. In certain embodiments, the protein equivalent source includes from about 25% to about 75% w/w of non-essential amino acids based on the total amino acids included in the protein equivalent source. In some embodiments, the protein equivalent source includes from about 40% to about 60% w/w of non-essential amino acids based on the total amino acids included in the protein equivalent source.

In some embodiments, the protein equivalent source includes leucine. In some embodiments, the protein equivalent source includes from about 2% to about 15% w/w leucine per the total amount of amino acids included in the protein equivalent source. In some embodiments, the protein equivalent source includes from about 4% to about 10% w/w leucine per the total amount of amino acids included in the protein equivalent source.

In some embodiments, the protein equivalent source includes lysine. In some embodiments, the protein equivalent source includes from about 2% to about 10% w/w lysine per the total amino acids included in the protein equivalent source. In some embodiments, the protein equivalent source includes from about 4% to about 8% w/w lysine per the total amino acids in the protein equivalent source.

In some embodiments, the protein equivalent source includes valine. In some embodiments, the protein equivalent source includes from about 2% to about 15% w/w valine per the total amino acids included in the protein equivalent source. In some embodiments, the protein equivalent source includes from about 4% to about 10% w/w valine per the total amino acids in the protein equivalent source.

In some embodiments, the protein equivalent source includes isoleucine. In some embodiments, the protein equivalent source includes from about 1% to about 8% w/w isoleucine per the total amino acids included in the protein equivalent source. In some embodiments, the protein equivalent source includes from about 3% to about 7% w/w isoleucine per the total amino acids in the protein equivalent source.

In some embodiments, the protein equivalent source includes threonine. In some embodiments, the protein equivalent source includes from about 1% to about 8% w/w threonine per the total amino acids included in the protein equivalent source. In some embodiments, the protein equivalent source includes from about 3% to about 7% w/w threonine per the total amino acids in the protein equivalent source.

In some embodiments, the protein equivalent source includes tyrosine. In some embodiments, the protein equivalent source includes from about 1% to about 8% w/w tyrosine per the total amino acids included in the protein equivalent source. In some embodiments, the protein equivalent source includes from about 3% to about 7% w/w tyrosine per the total amino acids in the protein equivalent source.

In some embodiments, the protein equivalent source includes phenylalanine. In some embodiments, the protein equivalent source includes from about 1% to about 8% w/w phenylalanine per the total amino acids included in the protein equivalent source. In some embodiments, the protein equivalent source includes from about 3% to about 7% w/w phenylalanine per the total amino acids in the protein equivalent source.

In some embodiments, the protein equivalent source includes histidine. In some embodiments, the protein equivalent source includes from about 0.5% to about 4% w/w histidine per the total amino acids included in the protein equivalent source. In some embodiments, the protein equivalent source includes from about 1.5% to about 3.5% w/w histidine per the total amino acids in the protein equivalent source.

In some embodiments, the protein equivalent source includes cysteine and/or cysteine. In some embodiments, the protein equivalent source includes from about 0.5% to about 4% w/w cysteine or cysteine per the total amino acids included in the protein equivalent source. In some embodiments, the protein equivalent source includes from about 1.5% to about 3.5% w/w cystine or cysteine per the total amino acids in the protein equivalent source.

In some embodiments, the protein equivalent source includes tryptophan. In some embodiments, the protein equivalent source includes from about 0.5% to about 4% w/w tryptophan per the total amino acids included in the protein equivalent source. In some embodiments, the protein equivalent source includes from about 1.5% to about 3.5% w/w tryptophan per the total amino acids in the protein equivalent source.

In some embodiments, the protein equivalent source includes methionine. In some embodiments, the protein equivalent source includes from about 0.5% to about 4% w/w methionine per the total amino acids included in the protein equivalent source. In some embodiments, the protein equivalent source includes from about 1.5% to about 3.5% w/w methionine per the total amino acids in the protein equivalent source.

In some embodiments, the protein equivalent source includes aspartic acid. In some embodiments, the protein equivalent source includes from about 7% to about 20% w/w aspartic acid per the total amino acids included in the protein equivalent source. In some embodiments, the protein equivalent source includes from about 10% to about 17% w/w aspartic acid per the total amino acids in the protein equivalent source.

In some embodiments, the protein equivalent source includes proline. In some embodiments, the protein equivalent source includes from about 5% to about 12% w/w proline per the total amino acids included in the protein equivalent source. In some embodiments, the protein equivalent source includes from about 7% to about 10% w/w proline per the total amino acids in the protein equivalent source.

In some embodiments, the protein equivalent source includes alanine. In some embodiments, the protein equivalent source includes from about 3% to about 10% w/w alanine per the total amino acids included in the protein equivalent source. In some embodiments, the protein equivalent source includes from about 5% to about 8% w/w alanine per the total amino acids in the protein equivalent source.

In some embodiments, the protein equivalent source includes glutamate. In some embodiments, the protein equivalent source includes from about 1.5% to about 8% w/w glutamate per the total amino acids included in the protein equivalent source. In some embodiments, the protein equivalent source includes from about 3% to about 6% w/w glutamate per the total amino acids in the protein equivalent source.

In some embodiments, the protein equivalent source includes serine. In some embodiments, the protein equivalent source includes from about 1.5% to about 8% w/w serine per the total amino acids included in the protein equivalent source. In some embodiments, the protein equivalent source includes from about 3% to about 5% w/w serine per the total amino acids in the protein equivalent source.

In some embodiments, the protein equivalent source includes arginine. In some embodiments, the protein equivalent source includes from about 2% to about 8% w/w arginine per the total amino acids included in the protein equivalent source. In some embodiments, the protein equivalent source includes from about 3.5% to about 6% w/w arginine per the total amino acids in the protein equivalent source.

In some embodiments, the protein equivalent source includes glycine. In some embodiments, the protein equivalent source includes from about 0.5% to about 6% w/w glycine per the total amino acids included in the protein equivalent source. In some embodiments, the protein equivalent source includes from about 1.5% to about 3.5% w/w glycine per the total amino acids in the protein equivalent source.

In some embodiments, the nutritional composition comprises between about 1 g and about 7 g of a protein equivalent source per 100 Kcal. In other embodiments, the nutritional composition comprises between about 3.5 g and about 4.5 g of protein equivalent source per 100 Kcal.

In some embodiments, the nutritional composition comprises between about 0.5 g/100 Kcal and about 2.5 g/100 Kcal of essential amino acids. In certain embodiments, the nutritional composition comprises between about 1.3 g/100 Kcal to about 1.6 Kcal of essential amino acids.

In some embodiments, the nutritional composition comprises between about 0.5 g/100 Kcal and about 2.5 g/100 Kcal of essential amino acids. In certain embodiments, the nutritional composition comprises between about 1.3 g/100 Kcal to about 1.6 Kcal of non-essential amino acids.

In some embodiments, the nutritional composition comprises from about 0.2 g/100 Kcal to about 0.5 g/100 Kcal of leucine. In some embodiments, the nutritional composition comprises from about 0.1 g/100 Kcal to about 0.4 g/100 Kcal of lysine. In some embodiments, the nutritional composition comprises from about 0.1 g/100 Kcal to about 0.4 g/100 Kcal of valine. In some embodiments, the nutritional composition comprises from about 0.08 g/100 Kcal to about 0.23 g/100 Kcal of isoleucine. In some embodiments, the nutritional composition comprises from about 0.08 g/100 Kcal to about 0.20 g/100 Kcal of threonine. In some embodiments, the nutritional composition comprises from about 0.10 g/100 Kcal to about 0.15 g/100 Kcal of tyrosine. In some embodiments, the nutritional composition comprises from about 0.05 g/100 Kcal to about 0.15 g/100 Kcal of phenylalanine. In some embodiments, the nutritional composition comprises from about 0.01 g/100 Kcal to about 0.09 g/100 Kcal of histidine. In some embodiments, the nutritional composition comprises from about 0.02 g/100 Kcal to about 0.08 g/100 Kcal of cystine or cysteine. In some embodiments, the nutritional composition comprises from about 0.02 g/100 Kcal to about 0.08 g/100 Kcal of tryptophan. In some embodiments, the nutritional composition comprises from about 0.02 g/100 Kcal to about 0.08 g/100 Kcal of methionine.

In some embodiments, the nutritional composition comprises from about 0.2 g/100 Kcal to about 0.7 g/100 Kcal of aspartic acid. In some embodiments, the nutritional composition comprises from about 0.1 g/100 Kcal to about 0.4 g/100 Kcal of proline. In some embodiments, the nutritional composition comprises from about 0.1 g/100 Kcal to about 0.3 g/100 Kcal of alanine. In some embodiments, the nutritional composition comprises from about 0.08 g/100 Kcal to about 0.25 g/100 Kcal of glutamate. In some embodiments, the nutritional composition comprises from about 0.08 g/100 Kcal to about 0.2 g/100 Kcal of serine. In some embodiments, the nutritional composition comprises from about 0.08 g/100 Kcal to about 0.15 g/100 Kcal of arginine. In some embodiments, the nutritional composition comprises from about 0.02 g/100 Kcal to about 0.08 g/100 Kcal of glycine.

The nutritional composition(s) of the present disclosure including the protein equivalent source, may be administered in one or more doses daily. Any orally acceptable dosage form is contemplated by the present disclosure. Examples of such dosage forms include, but are not limited to pills, tablets, capsules, soft-gels, liquids, liquid concentrates, powders, elixirs, solutions, suspensions, emulsions, lozenges, beads, cachets, and combinations thereof.

In some embodiments, the protein equivalent source may provide from about 5% to about 20% of the total calories for the nutritional composition. In some embodiments, the protein equivalent source may provide from about 8% to about 12% of the total calories for the nutritional composition.

Suitable fat or lipid sources for the nutritional composition of the present disclosure may be any known or used in the art, including but not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

Carbohydrate sources can be any used in the art, e.g., lactose, glucose, fructose, corn syrup solids, maltodextrins, sucrose, starch, rice syrup solids, and the like. The amount of carbohydrate in the nutritional composition typically can vary from between about 5 g and about 25 g/100 kcal. In some embodiments, carbohydrate source may be used in addition to the HMO and prebiotic components in the nutritional composition.

The nutritional composition(s) of the present disclosure may also comprise a carbohydrate source. Carbohydrate sources can be any used in the art, e.g., lactose, glucose, fructose, corn syrup solids, maltodextrins, sucrose, starch, rice syrup solids, and the like. The amount of carbohydrate in the nutritional composition typically can vary from between about 5 g and about 25 g/100 Kcal. In some embodiments, the amount of carbohydrate is between about 6 g and about 22 g/100 Kcal. In other embodiments, the amount of carbohydrate is between about 12 g and about 14 g/100 Kcal. In some embodiments, corn syrup solids are preferred. Moreover, hydrolyzed, partially hydrolyzed, and/or extensively hydrolyzed carbohydrates may be desirable for inclusion in the nutritional composition due to their easy digestibility. Specifically, hydrolyzed carbohydrates are less likely to contain allergenic epitopes.

In some embodiments, the nutritional composition described herein comprises a fat source. The enriched lipid fraction described herein may be the sole fat source or may be used in combination with any other suitable fat or lipid source for the nutritional composition as known in the art. In certain embodiments, appropriate fat sources include, but are not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

In some embodiment the nutritional composition comprises between about 1 g/100 Kcal to about 10 g/100 Kcal of a fat or lipid source. In some embodiments, the nutritional composition comprises between about 2 g/100 Kcal to about 7 g/100 Kcal of a fat source. In other embodiments the fat source may be present in an amount from about 2.5 g/100 Kcal to about 6 g/100 Kcal. In still other embodiments, the fat source may be present in the nutritional composition in an amount from about 3 g/100 Kcal to about 4 g/100 Kcal.

In some embodiments, the fat or lipid source comprises from about 10% to about 35% palm oil per the total amount of fat or lipid. In some embodiments, the fat or lipid source comprises from about 15% to about 30% palm oil per the total amount of fat or lipid. Yet in other embodiments, the fat or lipid source may comprise from about 18% to about 25% palm oil per the total amount of fat or lipid.

In certain embodiments, the fat or lipid source may be formulated to include from about 2% to about 16% soybean oil based on the total amount of fat or lipid. In some embodiments, the fat or lipid source may be formulated to include from about 4% to about 12% soybean oil based on the total amount of fat or lipid. In some embodiments, the fat or lipid source may be formulated to include from about 6% to about 10% soybean oil based on the total amount of fat or lipid.

In certain embodiments, the fat or lipid source may be formulated to include from about 2% to about 16% coconut oil based on the total amount of fat or lipid. In some embodiments, the fat or lipid source may be formulated to include from about 4% to about 12% coconut oil based on the total amount of fat or lipid. In some embodiments, the fat or lipid source may be formulated to include from about 6% to about 10% coconut oil based on the total amount of fat or lipid.

In certain embodiments, the fat or lipid source may be formulated to include from about 2% to about 16% sunflower oil based on the total amount of fat or lipid. In some embodiments, the fat or lipid source may be formulated to include from about 4% to about 12% sunflower oil based on the total amount of fat or lipid. In some embodiments, the fat or lipid source may be formulated to include from about 6% to about 10% sunflower oil based on the total amount of fat or lipid.

In some embodiments, the oils, i.e. sunflower oil, soybean oil, sunflower oil, palm oil, etc. are meant to cover fortified versions of such oils known in the art. For example, in certain embodiments, the use of sunflower oil may include high oleic sunflower oil. In other examples, the use of such oils may be fortified with certain fatty acids, as known in the art, and may be used in the fat or lipid source disclosed herein.

In some embodiments, the fat or lipid source includes an oil blend including sunflower oil, medium chain triglyceride oil, and soybean oil. In some embodiments, the fat or lipid source includes a ratio of sunflower oil to medium chain triglyceride oil of about 1:1 to about 2:1. In certain other embodiments, the fat or lipid source includes a ratio of sunflower oil to soybean oil of from about 1:1 to about 2:1. In still other embodiments, the fat or lipid source may include a ratio of medium chain triglyceride oil to soybean oil of from about 1:1 to about 2:1.

In certain embodiments the fat or lipid source may comprise from about 15% to about 50% w/w sunflower oil based on the total fat or lipid content. In certain embodiments, the fat or lipid source includes from about 25% to about 40% w/w sunflower oil based on the total fat or lipid content. In some embodiments, the fat or lipid source comprises from about 30% to about 35% w/w sunflower oil based on the total fat or lipid content.

In certain embodiments the fat or lipid source may comprise from about 15% to about 50% w/w medium chain triglyceride oil based on the total fat or lipid content. In certain embodiments, the fat or lipid source includes from about 25% to about 40% w/w medium chain triglyceride oil based on the total fat or lipid content. In some embodiments, the fat or lipid source comprises from about 30% to about 35% w/w medium chain triglyceride oil based on the total fat or lipid content.

In certain embodiments the fat or lipid source may comprise from about 15% to about 50% w/w soybean oil based on the total fat or lipid content. In certain embodiments, the fat or lipid source includes from about 25% to about 40% w/w soybean oil based on the total fat or lipid content. In some embodiments, the fat or lipid source comprises from about 30% to about 35% w/w soybean oil based on the total fat or lipid content.

In some embodiments, the nutritional composition comprises from about 1 g/100 Kcal to about 3 g/100 Kcal of sunflower oil. In some embodiments, the nutritional composition comprises from about 1.3 g/100 Kcal to about 2.5 g/100 Kcal of sunflower oil. In still other embodiments, the nutritional composition comprises from about 1.7 g/100 Kcal to about 2.1 g/100 Kcal of sunflower oil. The sunflower oil as described herein may, in some embodiments, include high oleic sunflower oil.

In certain embodiments, the nutritional composition if formulated to include from about 1 g/100 Kcal to about 2.5 g/100 Kcal of medium chain triglyceride oil. In other embodiments, the nutritional composition includes from about 1.3 g/100 Kcal to about 2.1 g/100 Kcal of medium chain triglyceride oil. Still in further embodiments, the nutritional composition includes from about 1.6 g/100 Kcal to about 1.9 g/100 Kcal of medium chain triglyceride oil.

In some embodiments, the nutritional composition may be formulated to include from about 1 g/100 Kcal to about 2.3 g/100 Kcal of soybean oil. In certain embodiments, the nutritional composition may be formulated to include from about 1.2 g/100 Kcal to about 2 g/100 Kcal of soybean oil. Still in certain embodiments, the nutritional composition may be formulated to include from about 1.5 g/100 Kcal to about 1.8 g/100 Kcal of soybean oil.

In some embodiments, the term “sunflower oil”, “medium chain triglyceride oil”, and “soybean oil” are meant to cover fortified versions of such oils known in the art. For example, in certain embodiments, the use of sunflower oil may include high oleic sunflower oil. In other examples, the use of such oils may be fortified with certain fatty acids, as known in the art, and may be used in the fat or lipid source disclosed herein.

In some embodiments, the fat or lipid source provides from about 35% to about 55% of the total calories of the nutritional composition. In other embodiments, the fat or lipid source provides from about 40% to about 47% of the total calories of the nutritional composition.

In certain embodiments the nutritional composition may be formulated, in some embodiments, such that from about 10% to about 23% of the total calories of the nutritional composition are provided by sunflower oil. In other embodiments, from about 13% to about 20% of the total calories in the nutritional composition may be provided by sunflower oil. Still, in other embodiments, from about 15% to about 18% of the total calories of the nutritional composition may be provided by sunflower oil.

In some embodiments, the nutritional composition may be formulated such that from about 10% to about 20% of the total calories are provided by MCT oil. In certain embodiments, from about 12% to about 18% of the total calories in the nutritional composition may be provided by MCT oil. Still, in certain embodiments, from about 14% to about 17% of the calories of the nutritional composition may be provided by MCT oil.

In some embodiments, the nutritional composition may be formulated such that from about 10% to 20% of the total calories of the nutritional composition are provided by soybean oil. In certain embodiments, from about 12% to about 18% of the total calories of the nutritional composition may be provided by soybean oil. In certain embodiments, from about 13% to about 16% of the total calories may be provided by soybean oil.

The nutritional composition of the present disclosure may comprise lactoferrin in some embodiments. Lactoferrins are single chain polypeptides of about 80 kD containing 1-4 glycans, depending on the species. The 3-D structures of lactoferrin of different species are very similar, but not identical. Each lactoferrin comprises two homologous lobes, called the N- and C-lobes, referring to the N-terminal and C-terminal part of the molecule, respectively. Each lobe further consists of two sub-lobes or domains, which form a cleft where the ferric ion (Fe3+) is tightly bound in synergistic cooperation with a (bi)carbonate anion. These domains are called N1, N2, C1 and C2, respectively. The N-terminus of lactoferrin has strong cationic peptide regions that are responsible for a number of important binding characteristics. Lactoferrin has a very high isoelectric point (˜pI 9) and its cationic nature plays a major role in its ability to defend against bacterial, viral, and fungal pathogens. There are several clusters of cationic amino acids residues within the N-terminal region of lactoferrin mediating the biological activities of lactoferrin against a wide range of microorganisms.

Lactoferrin for use in the present disclosure may be, for example, isolated from the milk of a non-human animal or produced by a genetically modified organism. The oral electrolyte solutions described herein can, in some embodiments comprise non-human lactoferrin, non-human lactoferrin produced by a genetically modified organism and/or human lactoferrin produced by a genetically modified organism.

Suitable non-human lactoferrins for use in the present disclosure include, but are not limited to, those having at least 48% homology with the amino acid sequence of human lactoferrin. For instance, bovine lactoferrin (bLF) has an amino acid composition which has about 70% sequence homology to that of human lactoferrin. In some embodiments, the non-human lactoferrin has at least 65% homology with human lactoferrin and in some embodiments, at least 75% homology. Non-human lactoferrins acceptable for use in the present disclosure include, without limitation, bLF, porcine lactoferrin, equine lactoferrin, buffalo lactoferrin, goat lactoferrin, murine lactoferrin and camel lactoferrin.

In some embodiments, the nutritional composition of the present disclosure comprises non-human lactoferrin, for example bLF. bLF is a glycoprotein that belongs to the iron transporter or transferring family. It is isolated from bovine milk, wherein it is found as a component of whey. There are known differences between the amino acid sequence, glycosylation patters and iron-binding capacity in human lactoferrin and bLF. Additionally, there are multiple and sequential processing steps involved in the isolation of bLF from cow's milk that affect the physiochemical properties of the resulting bLF preparation. Human lactoferrin and bLF are also reported to have differences in their abilities to bind the lactoferrin receptor found in the human intestine.

Though not wishing to be bound by this or any other theory, it is believed that bLF that has been isolated from whole milk has less lipopolysaccharide (LPS) initially bound than does bLF that has been isolated from milk powder. Additionally, it is believed that bLF with a low somatic cell count has less initially-bound LPS. A bLF with less initially-bound LPS has more binding sites available on its surface. This is thought to aid bLF in binding to the appropriate location and disrupting the infection process.

bLF suitable for the present disclosure may be produced by any method known in the art. For example, in U.S. Pat. No. 4,791,193, incorporated by reference herein in its entirety, Okonogi et al. discloses a process for producing bovine lactoferrin in high purity. Generally, the process as disclosed includes three steps. Raw milk material is first contacted with a weakly acidic cationic exchanger to absorb lactoferrin followed by the second step where washing takes place to remove nonabsorbed substances. A desorbing step follows where lactoferrin is removed to produce purified bovine lactoferrin. Other methods may include steps as described in U.S. Pat. Nos. 7,368,141, 5,849,885, 5,919,913 and 5,861,491, the disclosures of which are all incorporated by reference in their entirety.

In certain embodiments, lactoferrin utilized in the present disclosure may be provided by an expanded bed absorption (EBA) process for isolating proteins from milk sources. EBA, also sometimes called stabilized fluid bed adsorption, is a process for isolating a milk protein, such as lactoferrin, from a milk source comprises establishing an expanded bed adsorption column comprising a particulate matrix, applying a milk source to the matrix, and eluting the lactoferrin from the matrix with an elution buffer comprising about 0.3 to about 2.0 M sodium chloride. Any mammalian milk source may be used in the present processes, although in particular embodiments, the milk source is a bovine milk source. The milk source comprises, in some embodiments, whole milk, reduced fat milk, skim milk, whey, casein, or mixtures thereof.

In particular embodiments, the target protein is lactoferrin, though other milk proteins, such as lactoperoxidases or lactalbumins, also may be isolated. In some embodiments, the process comprises the steps of establishing an expanded bed adsorption column comprising a particulate matrix, applying a milk source to the matrix, and eluting the lactoferrin from the matrix with about 0.3 to about 2.0M sodium chloride. In other embodiments, the lactoferrin is eluted with about 0.5 to about 1.0 M sodium chloride, while in further embodiments, the lactoferrin is eluted with about 0.7 to about 0.9 M sodium chloride.

The expanded bed adsorption column can be any known in the art, such as those described in U.S. Pat. Nos. 7,812,138, 6,620,326, and 6,977,046, the disclosures of which are hereby incorporated by reference herein. In some embodiments, a milk source is applied to the column in an expanded mode, and the elution is performed in either expanded or packed mode. In particular embodiments, the elution is performed in an expanded mode. For example, the expansion ratio in the expanded mode may be about 1 to about 3, or about 1.3 to about 1.7. EBA technology is further described in international published application nos. WO 92/00799, WO 02/18237, WO 97/17132, which are hereby incorporated by reference in their entireties.

The isoelectric point of lactoferrin is approximately 8.9. Prior EBA methods of isolating lactoferrin use 200 mM sodium hydroxide as an elution buffer. Thus, the pH of the system rises to over 12, and the structure and bioactivity of lactoferrin may be comprised, by irreversible structural changes. It has now been discovered that a sodium chloride solution can be used as an elution buffer in the isolation of lactoferrin from the EBA matrix. In certain embodiments, the sodium chloride has a concentration of about 0.3 M to about 2.0 M. In other embodiments, the lactoferrin elution buffer has a sodium chloride concentration of about 0.3 M to about 1.5 M, or about 0.5 m to about 1.0 M.

In other embodiments, lactoferrin for use in the composition of the present disclosure can be isolated through the use of radial chromatography or charged membranes, as would be familiar to the skilled artisan.

The lactoferrin that is used in certain embodiments may be any lactoferrin isolated from whole milk and/or having a low somatic cell count, wherein “low somatic cell count” refers to a somatic cell count less than 200,000 cells/mL. By way of example, suitable lactoferrin is available from Tatua Co-operative Dairy Co. Ltd., in Morrinsville, New Zealand, from FrieslandCampina Domo in Amersfoort, Netherlands or from Fonterra Co-Operative Group Limited in Auckland, New Zealand.

Surprisingly, lactoferrin included herein maintains certain bactericidal activity even if exposed to a low pH (i.e., below about 7, and even as low as about 4.6 or lower) and/or high temperatures (i.e., above about 65° C., and as high as about 120° C.), conditions which would be expected to destroy or severely limit the stability or activity of human lactoferrin. These low pH and/or high temperature conditions can be expected during certain processing regimen for nutritional compositions of the types described herein, such as pasteurization. Therefore, even after processing regimens, lactoferrin has bactericidal activity against undesirable bacterial pathogens found in the human gut. The nutritional composition may, in some embodiments, comprise lactoferrin in an amount from about 25 mg/100 mL to about 150 mg/100 mL. In other embodiments lactoferrin is present in an amount from about 60 mg/100 mL to about 120 mg/100 mL. In still other embodiments lactoferrin is present in an amount from about 85 mg/100 mL to about 110 mg/100 mL.

In an embodiment, the nutritional composition(s) of the present disclosure comprises choline. Choline is a nutrient that is essential for normal function of cells. It is a precursor for membrane phospholipids, and it accelerates the synthesis and release of acetylcholine, a neurotransmitter involved in memory storage. Moreover, though not wishing to be bound by this or any other theory, it is believed that dietary choline and docosahexaenoic acid (DHA) act synergistically to promote the biosynthesis of phosphatidylcholine and thus help promote synaptogenesis in human subjects. Additionally, choline and DHA may exhibit the synergistic effect of promoting dendritic spine formation, which is important in the maintenance of established synaptic connections. In some embodiments, the nutritional composition(s) of the present disclosure includes about 40 mg choline per serving to about 100 mg per 8 oz. serving.

In an embodiment, the nutritional composition comprises a source of iron. In an embodiment, the source of iron is ferric pyrophosphate, ferric orthophosphate, ferrous fumarate or a mixture thereof and the source of iron may be encapsulated in some embodiments.

One or more vitamins and/or minerals may also be added in to the nutritional composition in amounts sufficient to supply the daily nutritional requirements of a subject. It is to be understood by one of ordinary skill in the art that vitamin and mineral requirements will vary, for example, based on the age of the subject. For instance, an infant may have different vitamin and mineral requirements than a child between the ages of one and thirteen years. Thus, the embodiments are not intended to limit the nutritional composition to a particular age group but, rather, to provide a range of acceptable vitamin and mineral components.

In certain embodiments, the composition may optionally include, but is not limited to, one or more of the following vitamins or derivations thereof: vitamin B1 (thiamin, thiamin pyrophosphate, TPP, thiamin triphosphate, TTP, thiamin hydrochloride, thiamin mononitrate), vitamin B2 (riboflavin, flavin mononucleotide, FMN, flavin adenine dinucleotide, FAD, lactoflavin, ovoflavin), vitamin B3 (niacin, nicotinic acid, nicotinamide, niacinamide, nicotinamide adenine dinucleotide, NAD, nicotinic acid mononucleotide, NicMN, pyridine-3-carboxylic acid), vitamin B3-precursor tryptophan, vitamin B6 (pyridoxine, pyridoxal, pyridoxamine, pyridoxine hydrochloride), pantothenic acid (pantothenate, panthenol), folate (folic acid, folacin, pteroylglutamic acid), vitamin B12 (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D3, 1,25,-dihydroxyvitamin D), vitamin E (α-tocopherol, α-tocopherol acetate, α-tocopherol succinate, α-tocopherol nicotinate, α-tocopherol), vitamin K (vitamin K1, phylloquinone, naphthoquinone, vitamin K2, menaquinone-7, vitamin K3, menaquinone-4, menadione, menaquinone-8, menaquinone-8H, menaquinone-9, menaquinone-9H, menaquinone-10, menaquinone-11, menaquinone-12, menaquinone-13), choline, inositol, β-carotene and any combinations thereof.

In other embodiments, the composition may optionally include, but is not limited to, one or more of the following minerals or derivations thereof: boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added to growing-up milks or to other children's nutritional compositions in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

In an embodiment, the children's nutritional composition may contain between about 10 and about 50% of the maximum dietary recommendation for any given country, or between about 10 and about 50% of the average dietary recommendation for a group of countries, per serving of vitamins A, C, and E, zinc, iron, iodine, selenium, and choline. In another embodiment, the children's nutritional composition may supply about 10 -30% of the maximum dietary recommendation for any given country, or about 10 -30% of the average dietary recommendation for a group of countries, per serving of B-vitamins. In yet another embodiment, the levels of vitamin D, calcium, magnesium, phosphorus, and potassium in the children's nutritional product may correspond with the average levels found in milk. In other embodiments, other nutrients in the children's nutritional composition may be present at about 20% of the maximum dietary recommendation for any given country, or about 20% of the average dietary recommendation for a group of countries, per serving.

The children's nutritional composition of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the present disclosure may optionally include one or more emulsifiers that may be added for stability of the final product. Examples of suitable emulsifiers include, but are not limited to, lecithin (e.g., from egg or soy), alpha lactalbumin and/or mono- and di-glycerides, and mixtures thereof. Other emulsifiers are readily apparent to the skilled artisan and selection of suitable emulsifier(s) will depend, in part, upon the formulation and final product. Indeed, the incorporation of HMO into a nutritional composition, such as an infant formula, may require the presence of at least on emulsifier to ensure that the HMO does not separate from the fat or proteins contained within the infant formula during shelf-storage or preparation.

In some embodiments, the nutritional composition may be formulated to include from about 0.5 wt % to about 1 wt % of emulsifier based on the total dry weight of the nutritional composition. In other embodiments, the nutritional composition may be formulated to include from about 0.7 wt % to about 1 wt % of emulsifier based on the total dry weight of the nutritional composition.

In some embodiments where the nutritional composition is a ready-to-use liquid composition, the nutritional composition may be formulated to include from about 200 mg/L to about 600 mg/L of emulsifier. Still, in certain embodiments, the nutritional composition may include from about 300 mg/L to about 500 mg/L of emulsifier. In other embodiments, the nutritional composition may include from about 400 mg/L to about 500 mg/L of emulsifier.

The nutritional compositions of the present disclosure may optionally include one or more preservatives that may also be added to extend product shelf life. Suitable preservatives include, but are not limited to, potassium sorbate, sodium sorbate, potassium benzoate, sodium benzoate, potassium citrate, calcium disodium EDTA, and mixtures thereof. The incorporation of a preservative in the nutritional composition including HMO ensures that the nutritional composition has a suitable shelf-life such that, once reconstituted for administration, the nutritional composition delivers nutrients that are bioavailable and/or provide health and nutrition benefits for the target subject.

In some embodiments the nutritional composition may be formulated to include from about 0.1 wt % to about 1.0 wt % of a preservative based on the total dry weight of the composition. In other embodiments, the nutritional composition may be formulated to include from about 0.4 wt % to about 0.7 wt % of a preservative based on the total dry weight of the composition.

In some embodiments where the nutritional composition is a ready-to-use liquid composition, the nutritional composition may be formulated to include from about 0.5 g/L to about 5 g/L of preservative. Still, in certain embodiments, the nutritional composition may include from about 1 g/L to about 3 g/L of preservative.

The nutritional compositions of the present disclosure may optionally include one or more stabilizers. Suitable stabilizers for use in practicing the nutritional composition of the present disclosure include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, and mixtures thereof. Indeed, incorporating a suitable stabilizer in the nutritional composition including HMO ensures that the nutritional composition has a suitable shelf-life such that, once reconstituted for administration, the nutritional composition delivers nutrients that are bioavailable and/or provide health and nutrition benefits for the target subject.

In some embodiments where the nutritional composition is a ready-to-use liquid composition, the nutritional composition may be formulated to include from about 50 mg/L to about 150 mg/L of stabilizer. Still, in certain embodiments, the nutritional composition may include from about 80 mg/L to about 120 mg/L of stabilizer.

The nutritional compositions of the disclosure may provide minimal, partial or total nutritional support. The compositions may be nutritional supplements or meal replacements. The compositions may, but need not, be nutritionally complete. In an embodiment, the nutritional composition of the disclosure is nutritionally complete and contains suitable types and amounts of lipid, carbohydrate, protein, vitamins and minerals. The amount of lipid or fat typically can vary from about 2 to about 7 g/100 kcal. The amount of protein typically can vary from about 1 to about 5 g/100 kcal. The amount of carbohydrate typically can vary from about 8 to about 14 g/100 kcal.

In some embodiments, the nutritional composition of the present disclosure is a growing-up milk. Growing-up milks are fortified milk-based beverages intended for children over 1 year of age (typically from 1-6 years of age). They are not medical foods and are not intended as a meal replacement or a supplement to address a particular nutritional deficiency. Instead, growing-up milks are designed with the intent to serve as a complement to a diverse diet to provide additional insurance that a child achieves continual, daily intake of all essential vitamins and minerals, macronutrients plus additional functional dietary components, such as non-essential nutrients that have purported health-promoting properties.

The exact composition of an infant formula or a growing-up milk or other nutritional composition according to the present disclosure can vary from market-to-market, depending on local regulations and dietary intake information of the population of interest. In some embodiments, nutritional compositions according to the disclosure consist of a milk protein source, such as whole or skim milk, plus added sugar and sweeteners to achieve desired sensory properties, and added vitamins and minerals. The fat composition is typically derived from the milk raw materials. Total protein can be targeted to match that of human milk, cow milk or a lower value. Total carbohydrate is usually targeted to provide as little added sugar, such as sucrose or fructose, as possible to achieve an acceptable taste. Typically, Vitamin A, calcium and Vitamin D are added at levels to match the nutrient contribution of regional cow milk. Otherwise, in some embodiments, vitamins and minerals can be added at levels that provide approximately 20% of the dietary reference intake (DRI) or 20% of the Daily Value (DV) per serving. Moreover, nutrient values can vary between markets depending on the identified nutritional needs of the intended population, raw material contributions and regional regulations.

The pediatric subject may be a child or an infant. For example, the subject may an infant ranging in age from 0 to 3 months, about 0 to 6 months, 0 to 12 months, 3 to 6 months, or 6 to 12 months. The subject may alternatively be a child ranging in age from 1 to 13 years, 1 to 6 years or 1 to 3 years. In an embodiment, the composition may be administered to the pediatric subject prenatally, during infancy, and during childhood.

In certain embodiments, disclosed herein are methods of stimulating the growth of gut bacteria in a subject via administration of the nutritional composition disclosed herein. Indeed, administration of the nutritional composition to a target subject stimulates the growth of certain species of gut bacteria including, Lactobacillus species, Bifidobacterium species, Allobaculum species, and/or combinations thereof. In some embodiment, in addition to stimulating the growth and amount of certain species of bacteria, administration of the nutritional composition disclose herein including HMO reduces the growth of harmful or pathogenic gut bacteria, such as certain Clostridium species in the gut of the subject. Accordingly disclosed herein are methods of establishing a beneficial gut bacteria profile in a pediatric subject, via administration of the nutritional composition disclosed herein.

Furthermore, in some embodiments administration of the nutritional compositions disclosed herein improve gut microbiota composition and/or activity. For example, administration of the nutritional composition herein to a target subject can increase the amount of beneficial bacteria such as Bifidobacterium species and Lactobacillus spp. In some embodiments, the nutritional compositions herein change the ratio of Firmicutes and bacteroides upon administration to a target subject. Further, in some embodiments, administration of the nutritional compositions disclosed herein may increase the amount of butyrate-producing bacteria.

Indeed, colonization of the human gut starts soon after birth and continues during the first year of life. Colonization of the gut, including the amount and types of bacteria, depend on multiple factors including dietary, environmental, and host factors. Indeed, after the first inoculation gut microbiota changes rapidly. Typically, the gut is dominated by Bifidobacteria species. Indeed, the gut microbiota plays a critical role in stimulating the maturation of the immune system, however, the composition of gut microbiota differs between breast-fed versus formula-fed infants. Indeed, the gut microbiota of breast-fed infants has been associated with a lower number of intestinal pathogens and less infectious diarrhea. Accordingly, without being bound by any particular theory, administration of the compositions disclosed herein may increase beneficial Bifidobacteria in formula-fed infants thus, providing health benefits. (See, for example, FIG. 3). Thus provided in certain embodiments herein, are methods for promoting a gut microbiota composition in a formula-fed infant that is more similar to a breast-fed infant via administration of the nutritional compositions disclosed herein to a formula-fed infant.

Further disclosed herein are methods for promoting cognitive development in the subject. More specifically, the present compositions and methods, in some embodiments, improve the normal mental performance, learning, memory, cognition and visual function in a subject. In other embodiments, the present compositions and methods support healthy, normal or improved behavioral, psychomotor and emotional development in a subject. In yet further embodiments, the present compositions and methods promote sensorimotor development, exploration and manipulation, object relatedness, visual acuity, objection recognition, visual attention and/or other aspects of cognitive processing.

While not being bound by any particular theory, several mechanisms of action may contribute to the beneficial gastrointestinal and neurological benefits of the nutritional compositions and methods of the present disclosure. For example, the compositions beneficial by products of gut microbiota may affect brain and influence behavior.

Further disclosed herein are methods for improving absorptive and digestive functions, including intestinal permeability, in target subjects by administering the nutritional composition disclosed herein to the target subject. Indeed, in some embodiments provided are methods for ameliorating the causes and symptoms of leaky gut in a target subject via administering the nutritional compositions disclosed herein to the target subject. Indeed, without being bound by any particular theory, administration of the nutritional compositions may target tight junction expression and cytokine production in the gastrointestinal tract, thus preventing and/or ameliorating the symptoms of leaky gut syndrome in the target individual. Further, administration of the disclosed nutritional compositions my lower the incidence of intestinal diarrhea.

Indeed, in pediatric subjects, especially newborn infants, the integrity of the epithelial layer is not complete and thus is subject to increased permeability to bacteria and luminal antigens that can trigger mucosal inflammation. Accordingly, administration of the nutritional composition disclosed herein addresses the issue of intestinal permeability by stimulating certain levels of tight junction (TJ) proteins and cytokines. Indeed, tight junction complexes are made up of complex lipoprotein structures. The tight junction is located at the most apical region of epithelial cells, and is composed of circumferential and continuous strands near the apex of the lateral membrane to serve as a molecular fence partitioning the cytosolic membrane into apical and basolateral domains. These tight junctions have more than 40 different proteins including occluding, claudin, and zonula occludens-1 (ZO-1), that participate in tight junction structural integrity by physically interacting with several tight junction proteins and by binding to the actin cytoskeleton. Pathogenic toxins and certain inflammatory mediators can alter expression and localization of tight junction proteins.

Accordingly, administration of the disclosed nutritional compositions, result in lower intestinal permeability thus, improving intestinal morphology and improving nutrient absorption. Further, administration of the disclosed nutritional composition can regulate expression and localization of tight junction proteins in the distal small intestines. Furthermore, administration of the nutritional composition disclosed herein can prevent redistribution of tight junction proteins into the cytoplasm. The redistribution of tight junction proteins from the intercellular junctions into the intracellular compartment is advantageous in preventing defective intestinal barrier function and in promoting intestinal barrier function.

Administration of the nutritional composition disclosed herein can beneficially impact short-chain fatty acid production and/or concentration in colon contents. For example, administration of the disclosed nutritional composition can stimulate the production of short-chain fatty acids produced by microbiota fermentation including acetate, propionate, and butyrate. Indeed, acetate and propionate can be absorbed into portal circulation, while butyrate can be used as an energy source for colonocytes by the host. The short-chain fatty acids produced by administration of the nutritional composition can function as signaling molecules and/or stimulate neurogenesis, and thus provide certain neuroprotective benefits to the target subject.

Furthermore, short-chain fatty acids can act via complementary mechanisms, i.e. butyrate acts via cAMP-dependent mechanisms while propionate acts via gut-brain neural circuit involving the fatty acid receptor FFAR3. Indeed, propionate is an agonist of FFAR3 in the periportal afferent neural system. Accordingly, administration of the nutritional composition disclosed herein that stimulates certain short-chain fatty acids can provide synergistic and complementary methods of action that provide increased and/or synergistic neuronal health benefits to the target subject.

Further provided herein are methods of manufacturing a nutritional composition, such as an infant formula, including at least one or a combination of the following steps: selecting a carbohydrate source, selecting a source of HMO, selecting a source of protein, selecting a source of fat, and combining the carbohydrate source, HMO, protein source and fat source to produce a nutritional composition. In some embodiments, the method further comprises the step of selecting certain amounts of each ingredient to be incorporated in specific amount based on a 100 kcal serving of the nutritional composition or based on the weight percentage of the nutritional composition.

In certain embodiments, administration of the nutritional compositions disclosed herein modify the microbiota in the lung and reduce the incidence of respiratory tract infections in target subjects. Indeed, the number and severity of respiratory tract infections, including human rhinovirus and respiratory syncytial virus, are the major cause of pathogen-related infant morbidity in developed and developing countries. Prematurity is a major risk factor for severe infection as premature infants are physiologically immune-deficient having immature innate immune system and thus, their stepwise compositional development of the gut and lung microbiota is disturbed. Accordingly, administering the nutritional compositions disclosed herein to infants, especially premature infants, can promote maturation of gut and lung microbiota and reduce the incidence and/or severity of respiratory tract infections.

Further, administration of the nutritional composition disclosed herein can reduce the risk of bacterial-associated respiratory tract infections, such as pneumonia. Indeed, acute lower respiratory tract infections caused by bacterial infection is also one of the leading causes of child mortality in developing countries. Streptococcus pneumoniae is a leading cause of bacterial pneumonia, meningitis, and sepsis in infants and children worldwide. Accordingly, administering the nutritional compositions disclosed herein can reduce the incidence and/or severity of bacterial pneumonia in children and infants.

Further, in some embodiments, administration of the nutritional compositions disclosed herein reduce inflammatory factors in lung tissue, such as inflammatory cytokines and chemokines, and further increase the production of mucosal IgA.

In some embodiments, provided herein are methods for improving overall health of a target subject via beneficial action in the gut, by administering the nutritional compositions disclosed herein, Indeed, in certain embodiments, administering the nutritional compositions disclosed herein containing the specific HMOs in addition to GOX and/or PDX, synergistically supports the growth of beneficial gut microbiota and promotes immune response.

Furthermore, without being bound by any particular theory, certain embodiments administration of the nutritional compositions disclosed herein may: (1) synergistically improve gut microbiota by increasing the levels of beneficial bacterial species, including Bifidobacterium longum infantis, Bifidobacterium longum longum, Bifidobacterium adolescenlis, Bifidobacterium breve, Bifidobacterium bifidum, Faecalibacterium prausnitzii, and/or Akkermansia municiphila (2) suppress the grown of pathogenic bacterial strains in the gut, for example Clostridium perfingens, Enterococcus faecalis, Escherichia coli, Streptococcus thermophilus, Citrobacter spp., and Clostridium difficile (3) lower microbiome alpha diversity measure (including richness and evenness) to resemble the gut microbiome of breast-fed infants (4) lower the exposure to antibiotic--resistant microbes, e.g. gamma-Proteobacteria (5) modulate the microbial production of short chain fatty acids produced by beneficial bacteria, such as butyrate, propionate, and/or acetate (6) enhance epithelia barrier function in the gut and improve immune system development and/or (7) promote the growth beneficial Clostridia (cluster XIVa and IV) thereby preventing the risk food allergy.

In some embodiments, provided herein are methods for accelerating, or supporting optimal brain development in target subjects, especially pediatric subjects_{;} by administering the nutritional compositions disclosed herein. Indeed, in certain embodiments, administering the nutritional compositions disclosed herein containing the specific HMOs in addition to GOX and/or PDX, synergistically accelerates and/or strengthens neurological connections and development in target subjects, such as pediatric subjects.

In some embodiments, the method is directed to manufacturing a powdered nutritional composition. The term “powdered nutritional composition” as used herein, unless otherwise specified, refers to dry-blended powdered nutritional formulations comprising protein, and specifically plant protein, and at least one of fat and carbohydrate, which are reconstitutable with an aqueous liquid, and which are suitable for oral administration to a human.

Indeed, in some embodiments, the method comprises the steps of dry-blending selected nutritional powders of the nutrients selected to create a base nutritional powder to which additional selected ingredients, such as HMO, may be added and further blended with the base nutritional powder. The term “dry-blended” as used herein, unless otherwise specified, refers to the mixing of components or ingredients to form a base nutritional powder or, to the addition of a dry, powdered or granulated component or ingredient to a base powder to form a powdered nutritional formulation. In some embodiments, the base nutritional powder is a milk-based nutritional powder. In some embodiments, the base nutritional powder includes at least one fat, one protein, and one carbohydrate. The powdered nutritional formulations may have a caloric density tailored to the nutritional needs of the target subject.

The powdered nutritional compositions may be formulated with sufficient kinds and amounts of nutrients so as to provide a sole, primary, or supplemental source of nutrition, or to provide a specialized powdered nutritional formulation for use in individuals afflicted with specific diseases or conditions. For example, in some embodiments, the nutritional compositions disclosed herein may be suitable for administration to pediatric subjects and infants in order provide exemplary health benefits disclosed herein.

The powdered nutritional compositions provided herein may further comprise other optional ingredients that may modify the physical, chemical, hedonic or processing characteristics of the products or serve as nutritional components when used in the targeted population. Many such optional ingredients are known or otherwise suitable for use in other nutritional products and may also be used in the powdered nutritional compositions described herein, provided that such optional ingredients are safe and effective for oral administration and are compatible with the essential and other ingredients in the selected product form. Non-limiting examples of such optional ingredients include preservatives, antioxidants, emulsifying agents, buffers, additional nutrients as described herein, colorants, flavors, thickening agents and stabilizers, and so forth.

The powdered nutritional compositions of the present disclosure may be packaged and sealed in single or multi-use containers, and then stored under ambient conditions for up to about 36 months or longer, more typically from about 12 to about 24 months. For multi-use containers, these packages can be opened and then covered for repeated use by the ultimate user, provided that the covered package is then stored under ambient conditions (e.g., avoid extreme temperatures) and the contents used within about one month or so.

In some embodiments, the method further comprises the step of placing the nutritional compositions in a suitable package. A suitable package may comprise a container, tub, pouch, sachet, bottle, or any other container known and used in the art for containing nutritional composition. In some embodiments, the package containing the nutritional composition is a plastic container. In some embodiments, the package containing the nutritional composition is a metal, glass, coated or laminated cardboard or paper container. Generally, these types of packaging materials are suitable for use with certain sterilization methods utilized during the manufacturing of nutritional compositions formulated for oral administration.

in some embodiments, the nutritional compositions are packaged in a container. The container for use herein may include any container suitable for use with liquid nutritional products that is also capable of withstanding aseptic processing conditions (e.g., sterilization) described herein and known to those of ordinary skill in the art. A suitable container may be a single-dose container, or may be a multi-dose resealable, or recloseable container that may or may not have a sealing member, such as a thin foil sealing member located below the cap. Non-limiting examples of such containers include bags, plastic bottles or containers, pouches, metal cans, glass bottles, juice box-type containers, foil pouches, plastic bags sold in boxes, or any other container meeting the above-described criteria. In some embodiments, the container is a. resealable multi-dose plastic container. In certain embodiments, the resealable multi-dose plastic container further comprises a foil seal and a plastic resealable cap. In some embodiments, the container may include a direct seal screw cap, In other embodiments, the container may be a flexible pouch,

In some embodiments, the nutritional composition is a liquid nutritional composition and is processed via a “retort packaging” or “retort sterilizing” process. The terms “retort packaging” and “retort sterilizing” are used interchangeably herein, and unless otherwise specified, refer to the common practice of filling a container, most typically a metal can or other similar package, with a nutritional liquid and then subjecting the liquid-filled package to the necessary heat sterilization step, to form a sterilized, retort packaged, nutritional liquid product.

In some embodiments, the nutritional compositions disclosed herein are processed via an acceptable aseptic packaging method. The term “aseptic packaging” as used herein, unless otherwise specified, refers to the manufacture of a packaged product without reliance upon the above-described retort packaging step, wherein the nutritional liquid and package are sterilized separately prior to filling, and then are combined under sterilized or aseptic processing conditions to form a sterilized, aseptically packaged, nutritional liquid product,

### EXAMPLES

Example 1 illustrates the synergy between human milk oligosaccharides (sialyllactose) and GOS/PDX in effect on metabolomics markers in fecal samples.

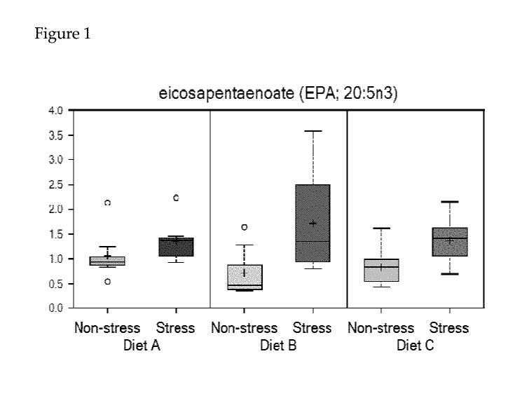

The goal of this study was to compare the metabolic profiles of mice to identify markers of stress and any changes in these markers clue to diet. In this study, we tested whether prebiotics (blend of galactooligosaccharides (GOS) and polydextrose (PDX)) and/or milk oligosaccharide sialyllactose (Si) support a normal metabolome in the presence of stress. Male C57BL/6 mice were placed on one of the experimental diets for 14d: A) Control, B) GOS+PDX [15 g/kg each]+SL [2.2g/kg] or C) SL [2.2 g/kg]. Metabolites in the colonic contents were assessed using LC/MS. Stressor-exposed mice fed diets enriched with prebiotics or milk oligosaccharides showed increases in polyunsaturated fatty acids (PUFAs) and endocannabinoids, compared with the non-stress groups. PUFAs are important as substrates for anti-inflammatory eicosanoids, such as the prostaglandins, leukotrienes and thromboxanes. The n3 PUFAs such as eicosapentaenoate (EPA) have anti-inflammatory properties (REF). In mice fed SAL+GOS/PDX. Stress mice's fecal samples had increased EPA levels (FIG. 1). This study demonstrates that dietary prebiotics and milk oligosaccharides can impact the colonic metabolome to potentially attenuate stressor-induced immunomodulation.

### Example 2

Example 2 illustrates the synergy between human milk oligosaccharides (sialyllactose) and GOS/PDX in effect on brain development.

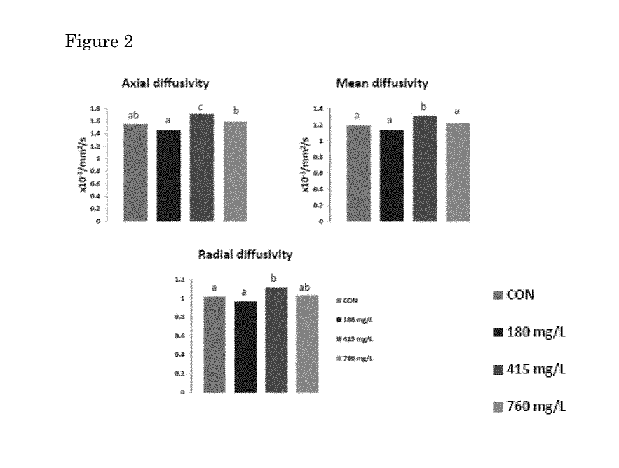

This study aimed to examine the dose response effects of sialyllactose (SL) in combination with galactooligosaccharides (GOS) and polydextrose (PDX) on piglet brain development. Beginning at 2 d of age, 38 (n=9-10 per treatment) vaginally-derived male piglets received one of four diets formulated to contain: control (CONT) [0 mg SL/L milk replacer], low (LOW) [180 mg SL/L], moderate (MOD) [415 mg SL/L], and high (HIGH) [760 mg SL/L]. All diets contained 4 g/L of a 1:1 mixture of PDX and GOS. At 32 or 33 d of age, piglets were subjected to magnetic resonance imaging (MRI) procedures to assess macrostructural and microstructural brain development. All outcomes were analyzed using a one-way ANOVA to assess differences between dietary treatments, significant outcomes were accepted at P≤0.05 and trends at 0.05<P<0.10. Axial, mean and radial diffusivity are used to assess white matter integrity and have been shown to change with age. Diffusion tensor imaging revealed differences due to dietary treatment in measures of axial diffusivity (P=0.002), mean diffusivity (P=0.004), and radial diffusivity (P=0.007) of the corpus callosum. In each of these outcomes, piglets provided the MOD diet exhibited the highest diffusivity measures compared with all other dietary treatments. Mean diffusivity (P=0.075) and radial diffusivity (P=0.051) in the left hippocampus tended to differ by dietary treatment, with piglets provided the MOD diet exhibiting the highest diffusivity measures. (See FIG. 2).

### FORMULATION EXAMPLES

Table 1 provides an example embodiment of a nutritional composition according to the present disclosure and describes the amount of each ingredient to be included per 100 kcal serving.

TABLE 1
Nutrition profile of an example nutritional composition

per 100 kcal

| Nutrient | Minimum | Maximum |
|---|---|---|
| Protein Equivalent Source (g) | 1.0 | 7.0 |
| Carbohydrates (g) | 6 | 22 |
| First Component HMO (g) | 0.015 | 2.2 |
| Second Component HMO (g) | 0.0015 | 1.2 |
| Prebiotic component containing | 0.1 | 1.0 |
| GOS (g) | | |
| Fat (g) | 1.3 | 7.2 |
| Prebiotic (g) | 0.3 | 1.2 |
| DHA (g) | 4 | 22 |
| Beta glucan (mg) | 2.9 | 17 |
| Probiotics (cfu) | 0.5 | 5.0 |
| Vitamin A (IU) | 9.60 × 10⁵ | 3.80 × 10⁸ |
| Vitamin D (IU) | 134 | 921 |
| Vitamin E (IU) | 22 | 126 |
| Vitamin K (mcg) | 0.8 | 5.4 |
| Thiamin (mcg) | 2.9 | 18 |
| Riboflavin (mcg) | 63 | 328 |
| Vitamin B6 (mcg) | 68 | 420 |
| Vitamin B12 (mcg) | 52 | 397 |
| Niacin (mcg) | 0.2 | 0.9 |
| Folic acid (mcg) | 690 | 5881 |
| Panthothenic acid (mcg) | 8 | 66 |
| Biotin (mcg) | 232 | 1211 |
| Vitamin C (mg) | 1.4 | 5.5 |
| Choline (mg) | 4.9 | 24 |
| Calcium (mg) | 4.9 | 43 |
| Phosphorus (mg) | 68 | 297 |
| Magnesium (mg) | 54 | 210 |
| Sodium (mg) | 4.9 | 34 |
| Potassium (mg) | 24 | 88 |
| Chloride (mg) | 82 | 346 |
| Iodine (mcg) | 53 | 237 |
| Iron (mg) | 8.9 | 79 |
| Zinc (mg) | 0.7 | 2.8 |
| Manganese (mcg) | 0.7 | 2.4 |
| Copper (mcg) | 7.2 | 41 |

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. For example, while methods for the production of a commercially sterile liquid nutritional supplement made according to those methods have been exemplified, other uses are contemplated. Therefore, the spirit and scope of the appended claims should not be limited to the description of the versions contained therein.

What is claimed is:

1. A nutritional composition comprising:
a protein or protein equivalent source;
a fat or lipid source;
a carbohydrate source;
a prebiotic comprising galacto-oligosaccharide;
a first human milk oligosaccharide component, comprising at least one human milk oligosaccharide or precursor thereof selected from the group consisting of 2′-fucosyllactose, 3′-fucosyllactose, 3′sialyllactose, 6′sialyllactose, lacto-N-biose, lacto-N-neotetraose, and lacto-N -tetraose; and
a second human milk oligosaccharide component, comprising at least one human milk oligosaccharide or precursor thereof selected from the group consisting of L-fucose and N -acetylglucosamine.

2. The nutritional composition of claim 1, wherein the galacto-oligosaccharides is present in an amount of from about 1 mg/mL to about 6 mg/mL.

3. The nutritional composition of claim 1, wherein the first HMO component is present in an amount of from about 0.5 mg/mL to about 10 mg/mL.

4. The nutritional composition of claim 1, wherein the second HMO component is present in an amount of from about 0.1 mg/mL to about 5 mg/mL.

5. The nutritional composition of claim 1, further comprising polydextrose.

6. The nutritional composition of claim 5, wherein the polydextrose is present in an amount ranging from about 0.1 g/100 kcal to about 0.5 g/00 kcal.

7. The nutritional composition of claim 1, further comprising βglucan.

8. The nutritional composition of claim 1, further comprising a probiotic.

9. The nutritional composition of claim 8, wherein the probiotic comprises Lactobacillus rhamnosus GG.

9. The composition of claim 8, wherein the probiotic is non-viable.

10. The nutritional composition of claim 8, wherein the probiotic is viable.

11. The nutritional composition of claim 9, wherein the Lactobacillus rhamnosus GG is present in an amount ranging from about 1×10⁵ cfu/100 kcals to about 1.5×10⁹ cfu/100 kcals.

12. The nutritional composition of claim 1, further comprising a source of long chain polyunsaturated fatty adds.

13. The nutritional composition of claim 12, wherein the source long chain polyunsaturated fatty adds comprises docosahexaenoic acid, arachidonic add, or a combination thereof.

14. The nutritional composition of claim 1, wherein the protein or protein equivalent source is present in an amount of from about 1 g/100 kcal to about 7 g/100 kcal; wherein the fat or lipid source is present in an amount of from about 1g/100 kcal to about 10 g/100 kcal; and wherein the carbohydrate source is present in an amount of from about 6 g/100 kcal to about 22 g/100 kcal.

15. The nutritional composition of claim 1, further comprising lactoferrin.

16. A method for modulating gut microbiota in an infant, comprising administering a nutritional composition comprising per 100 kcal:
(i) between about 1 g and about 7 g of a protein source,
(ii) between about 1 g and about 10 g of a lipid source,
(iii) between about 6 g and about 22 g of a carbohydrate source,
(iv) between about 0.015 g and about 2.2 g of a first human milk oligosaccharide component selected from the group consisting of 2′-fucosyllactose, 3′-fucosyllactose, 3′sialyllactose, 6′sialyllactose, lacto-N-biose, lacto-N-neotetraose, and lacto-N-tetraose,
(v) between about 0.0015 g and about 1.2 g of a second human milk oligosaccharide component selected from the group consisting of L-fucose, and N-acetylglucosamine, and
(vi) between about 0.1 g and 1.0 g of a galacto-oligosaccharide.

17. The method of claim 16, wherein the nutritional composition further comprises a probiotic.

18. The method of claim 17, wherein the probiotic comprises Lactobacillus rhamnosus GG.

19. A method for improving cognitive function and stimulating neuronal development in a target subject, comprising administering to the subject an effective amount of a nutritional composition comprising: (i) a protein source, (ii) a lipid source, (iii) a carbohydrate source, (iv) a first human milk oligosaccharide component selected from the group consisting of 2′-fucosyllactose, 3′-fucosyllactose, 3′sialyllactose, 6′sialyllactose, lacto-N-biose, lacto-N-neotetraose, and lacto-N -tetraose, (v) a second human milk oligosaccharide component selected from the group consisting of L-fucose and N-acetylglucosamine, and (vi) a prebiotic comprising galacto-oligosaccharide; wherein administration of the nutritional composition increasing the concentration of sialic acid in brain tissue of the target subject.

20. The method of claim 19, wherein the nutritional composition further comprises a probiotic.
